SEQUENCE LISTING (22 sequences)

SEQ ID NO: 1 - DNA, 365 bp, Artificial Sequence
  Description of Artificial Sequence Synthetic polynucleotide
gttttagagctagaaatagcaagttaaaataaggctagtccgttatcaacttgaaaaagtggcaccgagtcggtgcttttttataacttcgtatagcatacattatacgaagttatgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 2 - DNA, 510 bp, Artificial Sequence
  Description of Artificial Sequence Synthetic polynucleotide
tagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaattataacttcgtatagcatacattatacgaagttatgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 3 - DNA, 343 bp, Artificial Sequence
  Description of Artificial Sequence Synthetic polynucleotide
gttttagagctagaaatagcaagttaaaataaggctagtccgttatcaacttgaaaaagtggcaccgagtcggtgcttttttataacttcgtatagcatacattatacgaagttataattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccacagatcta

SEQ ID NO: 4 - DNA, 391 bp, Artificial Sequence
  Description of Artificial Sequence Synthetic polynucleotide
attcgaactcgaggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacggttttcttgctcttttttgtacgtctctgttttagagccggaaacggcaagttaaaataaggctagtccgttatcaacttgaaaaagtggcaccgagtcggtgcttttttgctagcgctaac

SEQ ID NO: 5 - DNA, 249 bp, Artificial Sequence
  Description of Artificial Sequence Synthetic polynucleotide
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 6 - DNA, 41 bp, Artificial Sequence
  Description of Artificial Sequence Synthetic primer
aatggactatcatatgcttaccgtaacttgaaagtatttcg

SEQ ID NO: 7 - DNA, 23 bp, Artificial Sequence
  Description of Artificial Sequence Synthetic primer
tattttaacttgccgtttccggc

SEQ ID NO: 8 - DNA, 33 bp, Artificial Sequence
  Description of Artificial Sequence Synthetic primer
acggactcgaggagggcctatttcccatgattc

SEQ ID NO: 9 - DNA, 43 bp, Artificial Sequence
  Description of Artificial Sequence Synthetic primer
gatcacggagctagcctgccatttgtctcaagatctagaattc

SEQ ID NO: 10 - DNA, 123 bp, Artificial Sequence
  Description of Artificial Sequence Synthetic primer
caccggagctggacggcgacgtaaagttttagagctagaaatagcaagttaaaataaggctagtccgttatcaacttgaaaaagtggcaccgagtcggtgcttgaagttcgagggcgacaccc

SEQ ID NO: 11 - DNA, 123 bp, Artificial Sequence
  Description of Artificial Sequence Synthetic primer
aaacgggtgtcgccctcgaacttcaagcaccgactcggtgccactttttcaagttgataacggactagccttattttaacttgctatttctagctctaaaactttacgtcgccgtccagctcc

SEQ ID NO: 12 - DNA, 131 bp, Artificial Sequence
  Description of Artificial Sequence Synthetic primer
caccggagctggacggcgacgtaaagttttagagctagaaatagcaagttaaaataaggctagtccgttatcaacttgaaaaagtggcaccgagtcggtgctttccccggggaagttcgagggcgacaccc

SEQ ID NO: 13 - DNA, 131 bp, Artificial Sequence
  Description of Artificial Sequence Synthetic primer
aaacgggtgtcgccctcgaacttccccggggaaagcaccgactcggtgccactttttcaagttgataacggactagccttattttaacttgctatttctagctctaaaactttacgtcgccgtccagctcc

SEQ ID NO: 14 - DNA, 28 bp, Artificial Sequence
  Description of Artificial Sequence Synthetic primer
agggatcctgagggcctatttcccatga

SEQ ID NO: 15 - DNA, 110 bp, Artificial Sequence
  Description of Artificial Sequence Synthetic primer
gcgctagctaaaaacagcatagctctaaaacgggtgtcgccctcgaacttcacagcatagctctaaaactttacgtcgccgtccagctcggtgtttcgtcctttccacaa

SEQ ID NO: 16 - DNA, 150 bp, Artificial Sequence
  Description of Artificial Sequence Synthetic primer
ttagcgctagctaaaagttttgggaccattcaaaacagcatagctctaaaacgggtgtcgccctcgaacttcgttttgggaccattcaaaacagcatagctctaaaactttacgtcgccgtccagctcggtgtttcgtcctttccacaag

SEQ ID NO: 17 - DNA, 182 bp, Artificial Sequence
  Description of Artificial Sequence Synthetic primer
agcgctagctaaaagttttgggaccattcaaaacagcatagctctaaaacgggtgtcgccctcgaacttcgttttgggaccattcaaaacagcatagctctaaaactttacgtcgccgtccagctcgttttgggaccattcaaaacagcatagctctaaaacggtgtttcgtcctttccaca

SEQ ID NO: 18 - DNA, 20 bp, Artificial Sequence
  Description of Artificial Sequence Synthetic oligonucleotide
gagctggacggcgacgtaaa

SEQ ID NO: 19 - DNA, 20 bp, Artificial Sequence
  Description of Artificial Sequence Synthetic oligonucleotide
gaagttcgagggcgacaccc

SEQ ID NO: 20 - DNA, 20 bp, Artificial Sequence
  Description of Artificial Sequence Synthetic oligonucleotide
gatcatccagctgtgacagg

SEQ ID NO: 21 - DNA, 20 bp, Artificial Sequence
  Description of Artificial Sequence Synthetic oligonucleotide
cctgtcacagctggatgatc

SEQ ID NO: 22 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

(19) United States
(12) Patent Application Publication
(10) Pub. No.: US 2020/0123533 A1
(43) Pub. Date: Apr. 23, 2020

(54) HIGH-THROUGHPUT STRATEGY FOR DISSECTING MAMMALIAN GENETIC INTERACTIONS

(71) Applicant: The Trustees of Columbia University in the City of New York, New York, NY (US)

(72) Inventors: Harris WANG, New York, NY (US); Sagi SHAPIRA, New York, NY (US); Victoria STOCKMAN, New York, NY (US)

(21) Appl. No.: 15/747,677

(22) PCT Filed: Aug. 1, 2016
(86) PCT No.: PCT/US16/44969

§ 371 Date: Jan. 25, 2018

Related U.S. Application Data
(60) Provisional application No. 62/199,291, filed on Jul. 31, 2015.

(51) Int. Cl. C12N 15/10; C12N 15/11; C12N 9/22

(52) U.S. Cl. CPC C12N 15/1068; C12N 15/11; C12N 2800/80; C12N 2310/20; C12N 9/22

(57) ABSTRACT

The present disclosure provides for a rapid and systematic method to map genetic interactions using the CRISPR/Cas system. A set of genes, such as the entire genome of an organism, can he targeted in pairs by a CRISPR guide RNA library. Each vector of the library contains at least two CRISPR guide sequences which encode gRNAs, The library can target all genes of the set in a pairwise fashion.

### CROSS REFERENCE TO RELATED APPLICATION

This application claims priority to U.S. Provisional Application No. 62/199,291 filed on Jul. 31, 2015, which is incorporated herein by reference in its entirety.

### GOVERNMENT LICENSE RIGHTS

This invention was made with government support under the National Institutes of Health (NIH) Grant Nos. 1DP5OD009172-02, 1U01GM110714-01A1, R01 GM109018-01 and U54 CA121852-07; National Science Foundation (NSF) Grant No. MCB-1453219, Defense Advanced Research Projects Agency (DARPA) Grant No. W911NF-15-2-0065, and Office of Naval Research (ONR) Grant No. N00014-15-1-2704. The government may have certain rights in the invention.

### FIELD OF THE INVENTION

The present invention relates to methods and systems for high-throughput combinatorial studies of genetic interactions. In particular, the present invention relates to a multiplex strategy for assessing genetic interactions using the Clustered Regularly Interspaced Short Palindromic Repeats (CRISPR)-Cas systems.

### BACKGROUND OF THE INVENTION

Complex cellular processes that control cell state and decision-making are orchestrated through highly interconnected regulatory networks. Quantitative genetic interaction mapping enables the systematic discovery of how gene-genetic interactions give rise to complex cellular processes [1]. Uncovering genetic interactions in lower organisms has led to novel insights into network topology and discovery of unexpected relationships between network components [2-4]. However, delineating these interactions has beer largely elusive in mammalian systems due to a lack of robust experimental tools. The CRISPR-Cas9 system enables efficient genome engineering of mammalian cells through a programmable guide-RNA (gRNA) that targets Cas9 to a desired locus for editing [5-8]. Thus far, studies using this system have focused on editing single loci [9-12] or multiple targets in select cases [13-15]. Recently, the CombiGEM approach was described to generate combinatorial gRNA libraries [16]. However, the approach requires iterative cloning steps and additional barcoding sequences. To extend CRISPR-Cas9 approaches for high-throughput combinatorial studies of genetic interactions, a general strategy is needed to interrogate pairs of chromosomal loci in a streamlined systematic and facile manner. Here, we describe the development of a multiplex strategy for assessing genetic interactions using CRISPR-Cas9.

### SUMMARY

The present disclosure provides for a method of constructing a guide RNA (gRNA) library (or a vector library) targeting a set of genes (or sequences). The method may comprise the following steps: (a) providing a plurality of forward primers and a plurality of reverse primers, each forward primer comprising at least one CRISPR guide sequence targeting at least one gene (or sequence) of the set of genes (or sequences), each reverse primer comprising at least one CRISPR guide sequence targeting at least one gene (or sequence) of the set of genes (or sequences), wherein the plurality of forward primers comprises CRISPR guide sequences targeting all genes (or sequences) of the set of genes (or sequences), wherein the plurality of reverse primers comprises CRISPR guide sequences targeting all genes (or sequences) of the set of genes (or sequences); and (b) conducting PCR reactions using the plurality of forward primers and the plurality of reverse primers.

The CRISPR guide sequence encodes a guide RNA (gRNA).

In one embodiment, the present method may target the set of genes (or sequences) in a pairwise fashion. Each forward primer may comprise one CRISPR guide sequence targeting one gene (or sequence) of the set of genes (or sequences); each reverse primer may comprise one CRISPR guide sequence targeting one gene (or sequence) of the set of genes (or sequences).

The present method may target the set of genes (or sequences) in a tri-wise fashion, in a quad-wise fashion, or in an n-wise fashion.

The method may further comprise a step (c), cloning the PCR products into a plurality of vectors. The vectors may be plasmids or viral vectors, such as lentiviral vectors. The vector may further comprise a selection marker and/or a reporter gene.

After the PCR products are cloned into vectors (e.g., step (c)), each vector may encode a CRISPR RNA (crRNA) and a trans-activating crRNA (tracrRNA), where the crRNA comprises the gRNA.

CrRNA and tracrRNA may be expressed as separate transcripts, or expressed as a single-guide RNA (sgRNA).

Expression of the CRISPR guide sequences may be under the control of U6 promoter, H1 promoter, T7 promoter, or a combination thereof.

The vector may further encode a Cas enzyme.

The set of genes (or sequences) may comprise the entire genome or a subset of the genome of an organism, such as a human, a mammal, etc.

The set of genes (or sequences) may comprise genes (or sequences) associated with one or more biological functions and/or one or more conditions or diseases. The set of genes (or sequences) may comprise genes (or sequences) associated with one or more cellular processes and/or one or more phenotypes. The set of genes (or sequences) may comprise genes (or sequences) associated with one or more cellular pathways.

The present disclosure also provides for a method of mapping genetic interactions by delivering into a population of cells a gRNA library (or a vector library) constructed by the present methods.

The cells may express a Cas enzyme. Alternatively or in addition, besides delivering the gRNA library to the cells, DNA or mRNA encoding a Cas enzyme is also delivered to the cells.

Non-limiting examples of the Cas enzymes include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, orthologs thereof, and modified versions thereof In one embodiment, the Cas enzyme is Cas9.

Expression of the Cas enzyme may be under the control of an inducible promoter or a constitutive promoter. The Cas enzyme may be wildtype or may comprise one or more mutations.

Also encompassed by the present disclosure are systems (e.g., gRNA libraries, vector libraries, etc.) constructed by the present method, a population of eukaryotic cells comprising the present systems (e.g., gRNA libraries, vector libraries, etc.) constructed by the present method, a kit comprising the present system (e.g., a gRNA library, vector libraries, etc.) constructed by the present method.

The present disclosure further provides for a system (e.g., a gRNA library, a vector library, etc.) targeting the entire genome or a subset of the genome of an organism in a pairwise fashion, in a tri-wise fashion, in a quad-wise fashion, or in an n-wise fashion. The library may comprise a plurality of vectors, wherein each vector comprises at least two CRISPR guide sequences that target at least two genes (or sequences) of the organism, and wherein the library targets (in parallel) every pair (or every group of three genes or sequences, or every group of four genes or sequences, or every group of n genes or sequences where n is a positive integer) of the genes (or sequences) of the entire genome or a subset of the genome of the organism.

The present method and/or the present system (e.g., a gRNA library, a vector library, etc.) may alter function of at least one gene (or sequence) of the set of genes (or sequences), or may alter function of all genes (or sequences) of the set of genes (or sequences), in a pairwise fashion, in a tri-wise fashion, in a quad-wise fashion, or in an n-wise fashion.

The present method and/or the present system (e.g., a gRNA library, a vector library, etc.) may alter expression of at least one gene (or sequence) of the set of genes (or sequences), or may alter function of all genes (or sequences) of the set of genes (or sequences), in a pairwise fashion, in a tri-wise fashion, in a quad-wise fashion, or in an n-wise fashion.

The present method and/or the present system (e.g., a gRNA library, a vector library, etc.) may decrease expression of at least one gene (or sequence) or all genes (or sequences) of the set of genes (or sequences) by CRISPR interference (CRISPRi), in a pairwise fashion, in a tri-wise fashion, in a quad-wise fashion, or in an n-wise fashion. The present method and/or the present system (e.g., the gRNA library) may increase expression of at least one gene (or sequence) of the set of genes (or sequences) by CRISPR activation (CRISPRa), in a pairwise fashion, in a tri-wise fashion, in a quad-wise fashion, or in an n-wise fashion.

The present method and/or the present system (e.g., a gRNA library, a vector library, etc.) may result in a knockout of at least one gene (or sequence) of the set of genes (or sequences). The present method and/or the present system (e.g., a gRNA library, a vector library, etc.) may result in a knockout of all genes (or sequences) of the set of genes (or sequences), in a pairwise fashion, in a tri-wise fashion, in a quad-wise fashion, or in an n-wise fashion.

Gene knockout may be confirmed by sequencing, such as next-generation sequencing (NGS).

### BRIEF DESCRIPTION OF THE DRAWINGS

FIG. 1. MoSAIC, a multiplex strategy for assessing genetic interactions using CRISPR-Cas9. (A) General strategy for generation of a combinatorial gRNA library targeting loci in a pairwise fashion. (B) Systematic pairwise interaction characterizations enabled by MoSAIC libraries contain gRNAs in both positions and single knockout controls. (C) Design of MoSAIC lentiviral expression systems for dual-transcript and single-transcript gRNAs.

FIG. 2. Characterization of dual U6 promoter MoSAIC designs. (A) Dual U6 promoter design tested in HEK293T, eGFP+ cells using an all-in-one vector, pLentcrispr_v1 containing both Cas9 and dual gRNA expression cassette. (B) SURVEYOR assay demonstrating positional bias at both eGFP and STAT1 targeted loci. Mv2 constructs used STAT1_gRNA_1. (C) Flow cytometry data at day 14 and 21 post viral transduction and puromycin selection. Indicated on the top-right of each plot are knockout (KO) percentage.

FIG. 3. Characterization of U6-H1 dual promoter MoSAIC designs (A) To test positional efficiency, eGFP gRNA_1 was placed in either position 1 (MV1.1, Mv5.1) or position 2 (MV1.2, MV5.2) and cloned into lentiviral vectors for testing Single gRNA vectors were tested (MV5.3) in comparison. (B) Positional eGFP knockout efficiency for various U6-H1 dual promoter designs was determined by flow cytometric analysis of eGFP expression at day 14 (bars without slanted lines). A separate experiment was done and eGFP KO efficiency was determined at day 28 (bars with slanted lines). Data shown with error-bars are averages from three independent experiments. Error-bars represent standard-errors (n=3).

FIG. 4. Characterization of single promoter, single transcript MoSAIC designs. (A) Schematic representation of gRNA lentivector backbone and gRNA transcript designs shown in complex with tracrRNA. MV3.2 contains only the first 12 bp of the 35 bp direct repeat (DR) sequence suggesting RNA cleavage is not necessary for Cas9 function. MV7.2 includes a spacer sequence TCCCCGGG (rationally designed to prevent hairpin formation) between gRNA sequences. gRNA1 is the eGFP gRNA_1 and gRNA 2 is eGFP gRNA_2. (B) Sequence of eGFP (SEQ ID NO. 22) that was targeted using gRNA1 (first boxed sequence) and gRNA2 (second boxed sequence). (C) Positional eGFP knockout efficiency was determined by flow cytometric analysis. All data shown are averages from three independent experiments. Error-bars represent standard-errors (n=3).

FIG. 5. Development of a Next-Generation Sequencing (NGS) optimized MoSAIC system. (A) Schematic representation of Cas9 with gRNA with sites altered shown as solid vertical bars. (B) Recovery of gRNA library for NGS. Left paid shows PCR recovery of gRNA barcodes from genomic DNA (short product indicated by bottom arrow, long product indicated by top arrow). Right panel shows undesired enrichment of short products during PCR amplification. (C) Design and efficiency characterization of MV6, the NGS optimized MoSAIC lentiviral system, at day 14 (bars without slanted lines). A separate experiment was done to characterize efficiencies at day 28 (bars with slanted lines). Data shown with error-bars are averages from three independent experiments. Error-bars represent standard-errors (n=3). (D) Crystal structure of Cas9 in complex with chimeric RNA variants The repeat and antirepeat sequences of the chimeric RNA form an RNA duplex, which interacts with the Rec1 domain on Cas9. Alterations in chimeric RNA v2 replace T-A Watson-Crick base pairing with C-G base pairing. Base pair alterations may stabilize RNA protein interactions, possibly increasing the efficiency of Cas9 activity. (E) Summary diagram of a lentiviral MoSAIC system for NGS quantification.

### DETAILED DESCRIPTION OF THE INVENTION

The present disclosure provides for a rapid and systematic method to map genetic interactions using the CRISPR/Cas system. A set of genes, such as the entire genome of an organism, can be targeted in pairs by a CRISPR gRNA library (e.g., encoded by DNA constructs such as vectors). Each vector of the library contains at least two CRISPR guide sequences which encode at least two gRNAs. The library can target all genes of interest in a pairwise fashion.

In order to comprehensively map genetic interactions in a gene network of a eukaryotic organism (e.g., a mammal), all possible single and double knockouts (KO) may be simultaneously interrogated. In one embodiment, the present method achieves this in a single step through polymerase chain reaction (PCR) of a common DNA template with CRISPR guide sequence primer pools. The first position CRISPR guide sequences (encoding gRNAs) act as the forward primers while the second position CRISPR guide sequences (encoding gRNAs) act as the reverse primers. The pooled PCR products ate then cloned into vectors (e.g., lentiviral expression vectors) resulting in an exhaustive combinatorial dual-gRNA library. In addition to directing genome editing to the desired targets, each CRISPR guide sequence pair in the library may serve as a unique molecular barcode of each mutant for subsequent multiplex interrogation of the cell population.

The method of constructing a guide RNA (gRNA) library (or a vector library encoding a gRNA library) targeting a set of genes (or sequences) may contain the following steps: (a) providing a plurality of forward primers and a plurality of reverse primers, each forward primer comprising at least one CRISPR guide sequence targeting at least one gene (or sequence) of the set of genes (or sequences), each reverse primer comprising at least one CRISPR guide sequence targeting at least one gene (or sequence) of the set of genes (or sequences), wherein the plurality of forward primers comprises CRISPR guide sequences targeting all genes (or sequences) of the set of genes (or sequences), wherein the plurality of reverse primers comprises CRISPR guide sequences targeting all genes (or sequences) of fie set of genes (or sequences), and wherein the CRISPR guide sequence encodes a guide RNA (gRNA); and (b) conducting polymerase chain reaction (PCR) using the plurality of forward primers and the plurality of reverse primers.

The methods are highly efficient for building large libraries for combinatorial genetic screening, and for parallel targeting of a great number of genomic loci. Accordingly, combinatorial sets of constructs such as the present libraries, may be used to catalog and map genetic factors associated with a diverse range of biological functions and diseases, and to identify genes and pathways that act synergistically to regulate a cellular process or phenotype.

The present systems and methods also encompass subsequent iterative introduction of gRNA-encoding constructs and enable higher-order combinatorial genetic perturbations. Pooled screening of multiple combination orders (e.g., pairwise, tri-wise, quad-wise, or n-wise combinations can be pooled and screened together simultaneously, where n is an integer) may be allowed. In addition, minimal combinations needed for a given application may be identified.

In an embodiment of the present method, the forward and reverse primers are used to amplify a template using, e.g., PCR. Any suitable sequence that may direct PCR using the forward and reverse primers may be used as a template, such as a DNA, a construct, a vector, etc.

The present constructs may contain at least two CRISPR guide sequences. The two or more CRISPR guide sequences may comprise two or more copies of a CRISPR guide sequence, two or more different CRISPR guide sequences, or combinations thereof.

The two or more CRISPR guide sequences may be operably linked to the same promoter or linked to different promoters. For example, the two or more CRISPR guide sequences may be operably linked to two or more promoters. In one embodiment, two CRISPR guide sequences are operably linked to two promoters; thus two transcripts comprising gRNAs would be transcribed. In another embodiment, two CRISPR guide sequences are operably linked to a promoter; thus one transcript (comprising a dual gRNA fusion) would be transcribed.

The two or more promoters may take any suitable position and/or orientation. For example, the two or more promoters may be unidirectional or bidirectional.

The forward primer and/or reverse primer may or may not contain at least one restriction site for cloning at a later stage. The restriction site can be specific to any suitable restriction enzymes, such as Type I, II or III restriction enzymes. Other types of restriction enzymes can also be used, including, but not limited to, Type IIS restriction endonucleases (e.g., Golden Gate Assembly, New England Biolabs).

The two CRISPR guide sequences may serve as barcodes that can then be PCR amplified and identified using next-generation sequencing.

The present invention generally relates to libraries, kits, methods, applications and screens used in functional genomics that focus on gene function in a cell which take advantage of the CRISPR-Cas systems. Every pair of genes in the genome of an organism may be knocked out in parallel by the present method. Also encompassed are methods of selecting cells with gene knockouts that survive under a selective pressure, methods of identifying the genetic basis of one or more disorders or diseases, and methods for constructing a genome-scale gRNA library.

CRISPR interference (CRISPRi) or CRISPR activation (CRISPRa) may be used in the present systems and methods.

CRISPRi is a transcriptional interference technique that allows for sequence-specific repression of gene expression and/or epigenetic modifications in cells. Qi et al., (2013) Repurposing CRISPR as an RNA-guided platform for sequence-specific control of gene expression. Cell 152 (5); 1173-83. CRISPRi regulates gene expression primarily on the transcriptional level. CRISPRi can sterically repress transcription, e.g., by blocking transcriptional initiation or elongation. The CRISPR guide sequence or gRNA may be complementary to the promoter and/or exonic sequences (such as the non-template strand and/or the template strand), and/or introns. Ji et al., (2014). Specific gene repression by CRISPRi system transferred through bacterial conjugation. ACS Synthetic Biology 3 (12): 929-31.

CRISPRi can also repress transcription via an effector domain. Fusing a repressor domain to a catalytically inactive Cas enzyme, e.g., dead Cas9 (dCas9), may further repress transcription. For example, the Krüppel associated box (KRAB) domain can be fused to dCas9 to repress transcription of the target gene. Gilbert et al., (2013). CRISPR-mediated modular RNA-guided regulation of transcription in eukaryotes. Cell 154 (2): 442-51.

CRISPRa utilizes the CRISPR technique to allow for sequence-specific activation of gene expression and/or epigenetic modifications in cells. Qi et al., (2013) Repurposing CRISPR as an RNA-guided platform for sequence-specific control of gene expression. Cell 152 (5): 1173-83. Gilbert et al., (2013) CRISPR-mediated modular RNA-guided regulation of transcription in eukaryotes, Cell 154 (2): 442-51. For example, a catalytically inactive Cas enzyme, e.g., dCas9, may be used to activate genes when fused to transcription activating factors. These factors include, but are not limited to, subunits of RNA Polymerase II and traditional transcription factors, such as VP16, VP64, VPR etc. Gilbert et al., 2014, Genome-Scale CRISPR-Mediated Control of Gene Repression and Activation, Cell 159 (3): 647-61.

The ability to downregulate or upregulate gene expression using the present system opens the door to large-scale genetic screens to uncover phenotypes that result from decreased or increased gene expression, in a pairwise fashion, in a tri-wise fashion, in a quad-wise fashion, or in an n-wise fashion. For example, the effects of gene downregulation and/or upregulation in cancer can be studied using the present method and system. Tanenbaum et al., (2014) A protein-tagging system for signal amplification in gene expression and fluorescence imaging, Cell 159 (3): 635-46.

### CRISPR Guide Sequence

In the CRISPR system, when the gRNA and the Cas enzyme are expressed, the gRNA directs sequence-specific binding of a CRISPR complex including a Cas enzyme to a target sequence (e.g., coding or non-coding DNA) in the cell. The Cas enzyme may then cleave the target sequence.

As used herein, a “CRISPR guide sequence” refers to a nucleic acid sequence that encodes a gRNA that is complementary to a target nucleic acid sequence in a host cell. The gRNA targets the CRISPR/Cas complex to a target nucleic acid sequence, also referred to as a target sequence or a target site.

As used herein, the term “gene” may also refer to a nucleic acid sequence; the term “genes” may also refer to nucleic acid sequences.

The gRNA may be between 10-30 nucleotides, 15-25 nucleotides, 15-20 nucleotides, 18-22 nucleotides, or 19-21 nucleotides in length. In some embodiments, a guide sequence is about or more than about 5, 10, 11, 12, 13, 14, 15, 16,17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In some embodiments, the gRNA is 20 nucleotides in length.

In general, a gRNA is any polynucleotide sequence having sufficient complementarity with a target sequence to hybridize with the target sequence and direct sequence-specific binding of a CRISPR complex to the target sequence. In some embodiments, the degree of complementarity between a gRNA and its corresponding target sequence, when optimally aligned using a suitable alignment algorithm, is about or more than about 50%, 55%, 60%, 65%, 70%, 75%, 80%, 85%, 90%, 95%, 96%, 97%, 98%, 99%, or 100%. In some embodiments, a gRNA is at least 50%, 55%, 60%, 65%, 70%, 75%, 80%, 85%, 90%, 95%, 96%, 97%, 98%, 99%, or at least 100% complementary to the 3′ end of the target sequence (e.g., the last 5, 6, 7, 8, 9, or 10 nucleotides of the 3′ end of the target sequence).

Differential gene expression can be achieved by modifying the efficiency of gRNA base-pairing to the target sequence. Larson et al., (2013). “CRISPR interference (CRISPRi) for sequence-specific control of gene expression”. Nature Protocols 8 (11): 2180-96. Modulating this efficiency may be used to create an allelic series for any given gene, creating a collection of hypomorphs and hypermorphs. These collections can be used to probe any genetic investigation. For hypomorphs, this allows the incremental reduction of gene function as opposed to the binary nature of gene knockouts.

Each construct or vector of the present system (e.g., a gRNA library, a vector library, etc.) may encode or contain two or more gRNAs. Multiple (two or more) gRNAs can be used to control multiple different genes simultaneously (multiplexing gene targeting), and/or to enhance the efficiency of regulating the same gene target.

The present DNA construct or vector may encode at least one CRISPR RNA (crRNA). CrRNA contains guide RNA along with a tracrRNA-binding segment which is complementary to at least one portion of a tracrRNA and functions to bind (hybridize to) the tracrRNA and recruit the Cas enzyme to the target sequence.

A tracrRNA-binding segment includes any sequence that has sufficient complementarity with tracrRNA to promote one or more of: (1) excision of a target sequence targeted by gRNA; and (2) formation of a CRISPR complex at or near a target sequence.

In some embodiments, the degree of complementarity between tracrRNA and tracrRNA-binding segment is about or more than about 25%, 30%, 40%, 50%, 60%, 65%, 70%, 75%, 80%, 85%, 90%, 95%, 97.5%, 99%, or 100%, along the length of the shorter of the two when optimally aligned, e.g., over a stretch of at least 8 contiguous, at least 9 contiguous, at least 10 contiguous, at least 11 contiguous, at least 12 contiguous, at least 13 contiguous, at least 14 contiguous or at least 15 contiguous nucleotides.

Exemplary tracrRNA-binding segment sequences can be found, for example, in Jinek, et al. Science (2012) 337(6096):816-821; Ran, et al. Nature Protocols (2013) 8:2281-2308; WO2014/093694; WO2013/176772 and WO2016070037.

TracrRNA-binding segment sequences may be wildtype or mutated.

“Complementarity” refers to the ability of a nucleic acid to form hydrogen bond(s) with another nucleic acid sequence by either traditional Watson-Crick base pairing or other non-traditional types of pairing. “Substantially complementary” refers to a degree of complementarity that is about or more than about 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides (e.g., contiguous nucleotides), or refers to two nucleic acids that hybridize under stringent conditions. As used herein, “stringent conditions” for hybridization refers to conditions under which a nucleic acid having complementarity to a target sequence predominantly hybridizes with the target sequence, and substantially does not hybridize to non-target sequences. Stringent conditions are generally sequence-dependent, and vary depending on a number of factors. Non-limiting examples of stringent conditions are described in detail in Tijssen (1993), Laboratory Techniques in Biochemistry and Molecular Biology-Hybridization with Nucleic Acid Probes, Part 1, Second Chapter “Overview of principles of hybridization and the strategy of nucleic acid probe assay”, Elsevier, N.Y.

The present constructs may or may not contain barcode elements. Barcode elements may be used as identifiers for a construct and may indicate the presence of one or more specific CRISPR guide sequences in a construct (e.g., a vector, DNA of cells introduced with the present constructs). Members of a set of barcode elements have a sufficiently unique nucleic acid sequence such that each barcode element is readily distinguishable from the other barcode elements of the set. Barcode elements may be any length of nucleotide, e.g., 6, 7, 8, 9, 10, 11, 12, 13, 14, 15, 16, 17, 18, 19, 20, 21, 22, 23, 25, 26, 27, 28, 29, or 30 or more nucleotides in length. Detecting barcode elements and determining the nucleic acid sequence of a barcode element or plurality of barcode elements are used to determine the presence of an associated DNA element of a genetic construct. Barcode elements can be detected by any method known in the art, including sequencing or microarray methods.

In one embodiment, the CRISPR guide sequences may serve as barcode elements.

tracrRNA

The present system (a construct or a vector) may contain or encode a tracrRNA.

A trans-activating crRNA (tracrRNA) refers to a RNA that recruits a Cas enzyme to a target sequence bound (hybridized) to a complementary crRNA.

In some embodiments, the tracrRNA is about or more than about 5, 6, 7, 8, 9, 10, 11, 12, 13, 14, 15, 16, 17, 18, 19, 20, 25, 26, 30, 32, 40, 45, 48, 50, 54, 63, 67, 85, or more nucleotides in length.

In some embodiments, the tracrRNA has sufficient complementarity to a tracrRNA-binding segment of crRNA to hybridize and participate in formation of a CRISPR complex.

TracrRNA sequences may be wildtype or mutated.

sgRNA

In some embodiments, crRNA (containing gRNA) and tracrRNA are expressed as separate transcripts. The present system (a construct or a vector) may contain or express crRNA and tracrRNA as separate transcripts.

In another embodiment, crRNA (containing gRNA and tracrRNA-binding segment) and tracrRNA are contained within a single transcript (e.g., sgRNA). The present system (a construct or a vector) may contain or express sgRNA.

A single guide RNA (sgRNA) is a chimeric RNA containing a tracrRNA and at least one crRNA (containing gRNA). An sgRNA has the dual function of both binding (hybridizing) to a target sequence and recruiting the Cas enzyme to the target sequence.

CrRNA and tracrRNA can be covalently linked via the 3′ end of the crRNA and the 5′ end of the tracrRNA. Alternatively, crRNA and tracrRNA can be covalently linked via the 5′ end of the crRNA and the 3′ end of the tracrRNA.

In such embodiments, sgRNA may have a secondary structure, such as a hairpin. In certain embodiments, the transcript or transcribed polynucleotide sequence has at least two or more hairpins. For Example, the transcript may have two, three, four, five, or more than five hairpins. SgRNA may comprise a linker loop stricture and/or a stem-loop structure. sgRNA used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In some embodiments, sgRNA can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate sgRNA design, many computational tools have been developed (See Prykhozhij et al. (PLoS ONE, 10(3): (2015)); Zhu et al. (PLoS ONE, 9(9) (2014)); Xiao et al. (Bioinformatics. January 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there is a publically available software tool that can be used to facilitate the design of sgRNA(s) (http://www.genscript.com/gRNA-design-tool.html).

SgRNA sequences may be wildtpe or mutated.

Chimeric RNA may be used to refer to a fusion of at least the tracrRNA-binding segment and tracrRNA. Chimeric RNA sequences may be wildtype or mutated.

### Cas Enzymes

The present system (e.g., constructs, vectors, cells, etc.) may or may not encode a Cas enzyme.

The Cas enzyme directs cleavage of one or two strands at or near a target sequence, such as within the target sequence and/or within the complementary strand of the target sequence. For example, the Cas enzyme may direct cleavage of one or both strands within about 1, 2, 3, 4, 5, 6, 7, 8, 9, 10, 15, 20, 25, 50, 100, 200, 500, or more nucleotides from the first or last nucleotide of a target sequence. In certain embodiments, format on of a CRISPR complex results in cleavage (e.g., a cutting or nicking) of one or both strands in or near (e.g. within 1, 2, 3, 4, 5, 6, 7, 8, 9, 10, 20, 50, or more base pairs from) the target sequence. In some embodiments, the Cas enzyme lacks DNA strand cleavage activity.

The Cas enzyme may be a type II, type I, type III, type IV or type V CRISPR system enzyme. In some embodiments, the Cas enzyme is a Cas9 enzyme (also known as Csn1 and Csx12). Non-limiting examples of the Cas9 enzyme include Cas9 derived from Streptococcus pyogenes (S. pyogenes), S. pneumoniae, Staphylococcus aureus, Neisseria meningitidis, Streptococcus thermophilus (S. thermophilus), or Treponema denticola. The Cas enzyme may also be derived from Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter.

Non-limiting examples of the Cas enzymes also include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, orthologs thereof, or modified versions thereof.

One or more of the CRISPR guide sequences and a Cas enzyme may be encoded by the same construct (e.g., a vector). Alternatively, a Cas enzyme may be encoded by a construct (e.g., a vector) separate from the vector encoding gRNAs. In some embodiments, the present system comprises two or more Cas enzyme coding sequences operably linked to different promoters. In some embodiments, the host cell expresses one or more Cas enzymes.

The Cas enzyme can be introduced into a cell in the form of a DNA, mRNA or protein. The Cas enzyme may be engineered, chimeric, or isolated from an organism.

Wildtype or mutant Cas enzyme may be used. In some embodiments, the nucleotide sequence encoding the Cas9 enzyme is modified to alter the activity of the protein. The mutant Cas enzyme may lack the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that tender Cas9 a nickase include, without limitation, D10A, H840A, N854A, N863A, and combinations thereof. In some embodiments, a Cas9 nickase may be used in combination with guide RNA(s), e.g., two guide RNAs, which target respectively sense and antisense strands of the DNA target.

Two or more catalytic domains of Cas9 (RuvC and/or HNH domains) may be mutated to produce a mutated Cas9 substantially lacking all DNA cleavage activity (a catalytically inactive Cas9). In some embodiments, a D10A mutation is combined with one or more of H840A, N854A, or N863A mutations to produce a Cas9 enzyme substantially lacking DNA cleavage activity (dead Cas 9 or dCas9). In some embodiments, a Cas enzyme is considered to substantially lack DNA cleavage activity when the DNA cleavage activity of the mutated enzyme is about or less than about 25%, 10%, 5%, 1%, 0.1%, 0.01%, or lower, compared to its non-mutated (wildtype) form. Other mutations may be useful; where the Cas9 or other Cas enzyme is from a species other than S. pyogenes, mutations in corresponding amino acids may be made to achieve similar effects.

Another Cas enzyme, Cpf1 (Cas protein 1 of PreFran subtype) may also be used in the present systems and methods. Zetsche et al. Cell, 163 (3): 759-771. In one embodiment, CRISPR-Cpf1 system can be used to cleave a desired region at or near a target sequence. A Cpf1 nuclease may be derived from Pwvetella spp., Francisella spp., etc.

Alternatively or in addition, the Cas enzyme may be fused to another protein or portion thereof. In some embodiments, dCas9 is fused to a repressor domain, such as a KRAB domain. In some embodiments, such dCas9 fusion proteins are used with the constructs described herein for multiplexed gene repression (e.g., CRISPR interference (CRISPRi)). In some embodiments, dCas9 is fused to an activator domain, such as VP64 or VPR. In some embodiments, such dCas9 fusion proteins are used with the constructs described herein for multiplexed gene activation (e.g. CRISPR activation (CRISPRa)).

In some embodiments, dCas9 is fused to an epigenetic modulating domain, such as a histone demethylase domain or a histone acetyltransferase domain. In some embodiments, dCas9 is fused to a LSD1 or p300, or a portion thereof. In some embodiments, the dCas9 fusion is used for CRISPR-based epigenetic modulation.

In some embodiments, dCas9 or Cas9 is fused to a Fokl nuclease domain In some embodiments, Cas9 or dCas9 fused to a Fokl nuclease domain is used for multiplexed gene editing.

In some embodiments, Cas9 or dCas9 is fused to a fluorescent protein (e.g., GFP, RFP, mCherry, etc), for, e.g., multiplexed labeling and/or visualization of genomic loci.

A sequence encoding a Cas enzyme may be codon-optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amine acid sequence. In some embodiments, one or more codons (e.g. 1, 2, 3, 4, 5, 10, 15, 20, 25, 50, or more, or all codons) in a sequence encoding a Cas enzyme correspond to the most frequently used codon for a particular amino acid in the host cell.

The Cas enzyme may be part of a fusion protein comprising one or more heterologous protein domains (e.g. about or more than about 1, 2, 3, 4, 5, 6, 7, 8, 9, 10, or more domains in addition to the Cas enzyme). A Cas enzyme fusion protein may comprise any additional protein sequence, and optionally a linker sequence between any two domains. Examples of protein domains that may be fused to a Cas enzyme include, without limitation, epitope tags, reporter proteins (or reporters), and protein domains having one or more of the following activities methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). The sequence encoding a Cas enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV; BP 16 protein fusions. U.S. Patent Publication No. 20110059502, WO2015065964. In some embodiments, a tagged Cas enzyme is used to identify the location of a target sequence.

The Cas enzyme may contain one or more nuclear localization sequences (NLS).

The present construct (e.g., a vector) may contain one, two or more enzyme-coding sequences. The two or more enzyme-coding sequences may comprise two or more copies of a single enzyme-coding sequence, two or more different enzyme-coding sequences, or combinations of these. In such an arrangement, the two or more enzyme-coding sequences may be operably linked to a promoter or to different promoters in a single vector or in multiple vectors. For example, a single vector, or multiple vectors, may comprise about or more than about 1, 2, 3, 4, 5, 6, 7, 8, 9, 10, 15, 20, or more enzyme-coding sequences. In some embodiments, about or more than about 1, 2, 3, 4, 5, 6, 7, 8, 9, 10, or more such enzyme-coding sequence-containing vectors may be provided, and optionally delivered to a cell.

### Target Sequences

A target sequence refers to any nucleic acid sequence in a host cell that may be targeted by the present systems. A CRISPR guide sequence may be selected to target any target sequence. The target sequence may be a sequence within a genome of an organism.

In certain embodiments, the target sequence is flanked downstream (on the 3′ side) by a protospacer adjacent motif (PAM). The sequence and length requirements for the PAM differ depending on the Cas enzyme used. PAMs may be 2-8 base pair sequences adjacent the target sequence. For example, for Cas9 endonucleases derived from Streptococcus pyogenes (S. pyogenes), the PAM sequence is NGG. For Cas9 endonucleases derived from Staphylococcus aureus, the PAM sequence is NNGRRT. For Cas9 endonucleases derived from Neisseria meningitidis, the PAM sequence is NNNNGATT. For Cas9 endonucleases derived from Streptococcus thermophilus, the PAM sequence is NNAGAA. For Cas9 endonuclease derived from Treponema denticola, the PAM sequence is NAAAAC. For a Cpf1 nuclease, the PAM sequence is TTN.

A target sequence may be located in the nucleus or cytoplasm of a cell. The target sequence may be within an organelle of a eukaryotic cell, for example, mitochondrion or chloroplast. The target sequence can be a sequence encoding a gene product (e.g., a protein) or a non-coding sequence (e.g., a regulatory polynucleotide or a junk DNA). A target sequence may be endogenous (endogenous to the cell) or exogenous (exogenous to the cell) sequences. A target sequence may he genomic nucleic acid and/or extra-genomic nucleic acid.

Target sequences may be nucleic acids encoding transcription factors, signaling proteins, transporters, epigenetic genes, etc. Target sequences may be, or contain part(s) of, constitutive exons downstream of a start codon of a gene. The target sequences may be, or contain part(s) of, either a first or a second exon of a gene. In one embodiment, the target sequence is a transcribed or non-transcribed strand of a gene.

Target sequences may be gene regulator) sequences such as promoters and transcriptional enhancer sequences, ribosomal binding sites and other sites relating to the efficiency of transcription, translation, or RNA processing, as well as coding sequences that control the activity, post-translational modification, or turnover of the encoded proteins. U.S. Patent Publication No. 20160186168.

Target sequences may include a number of disease-associated genes and polynucleotides as well as signaling pathway-associated genes and polynucleotides. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

The set of genes targeted by the present system and method may be the entire genome of an organism, or may be a subset of the genome of an organism. The set of genes may relate to a particular pathway (for example, an enzymatic pathway, an immune pathway or a cell division pathway), or a particular disease or group of diseases or disorders (e.g., cancer). U.S. Patent Publication No. 20150064138.

For example, the set of genes may be a group of genes associated with epigenetic changes in cancer, diabetes, obesity, neurological disorders (e.g., schizophrenia), or function in processes such as aging, http://www.epigenesys.eu/en/articles/features/638-what-is-epigenetics?showall=1.

In one embodiment, a set of genes refers to any of the clusters of genes described in Kazuhiro et al., epigenetic clustering of gastric carcinomas based on DNA methylation profiles at the precancerous stage: its correlation with tumor aggressiveness and patient outcome. Carcinogenesis, 2015, Vol. 36, No. 5, 500-520.

The set of genes may be a set of about or more than about 5, 10, 20, 50, 100, 200, 300, 400, 500, 600, 700, 800, 900, 1000, 10000, 20000 . . . n genes, or the entire genome of an organism.

The target sequences may be different loci within the same gene(s). The target sequences may be different genes. The present library may target 2 to 60 different loci within the same gene target or across multiple gene targets. For example, the present library may target 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 different target sequences (the set of genes). In some embodiments, the present library may target more than 60 different loci within the same gene target or across multiple gene targets, such as 65, 70, 75, 80, 85, 90, 95, 100 or more different DNA sequences (the set of genes).

### Regulatory Elements Including Promoters

The present system may contain one or more regulatory elements that are operably linked to one or more elements of the present CRISPR system so as to drive expression of the one or more elements of the present CRISPR system.

Regulatory elements may include promoters, enhancers, activator sequences, and other expression control elements (e.g. transcription termination signals, such as polyadenylation signals and poly-U sequences). The vectors of the invention may optionally include 5′ leader or signal sequences. Such regulatory elements are described, for example, in Goeddel, Gene Expression Technology: Methods in Enzymology, Academic Press (1990). A tissue-specific promoter may direct expression primarily in a desired tissue of interest. Regulatory elements may direct expression in a tissue-specific, cell-type specific, and/or a temporal-dependent manner, such as in a cell-cycle dependent or developmental stage-dependent manner.

In some embodiments, a vector comprises one or more pol III promoter (e.g. 1, 2, 3, 4, 5, or more pol III promoters), one or more pol II promoters (e.g. 1, 2, 3, 4, 5, or more pol II promoters), such as a mammalian RNA polymerase II promoter, one or more pol I promoters (e.g. 1, 2, 3 ,4, 5, or more pol I promoters), or combinations thereof.

Examples of pol III promoters include, bit are not limited to, H1 promoter, U6 promoter, mouse U6 promoter, swine U6 promoter. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV4G promoter, the dihydro folate reductase promoter, the β-actin promoter, the phosphoglycerol kinase (PGK) promoter, and the EF1 a promoter. Boshart et al. Cell, 41 :521-530 (1985). In some embodiments, the promoter is a human ubiquitin C promoter (UBCp). In some embodiments, the promoter is a viral promoter. In some embodiments, the promoter is a human cytomegalovirus promoter (CMVp).

Non-limiting examples of enhancers include WPRE; CMV enhancers; the R-U5′ segment in LTR of HTLV-1 (Mol. Cell. Biol Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

The present vector may contain one or more promoters upstream of the CRISPR guide sequence, the sequence encoding crRNA, the sequence encoding tracrRNA, the sequence encoding sgRNA, the sequence encoding the chimeric RNA (containing tracrRNA-binding segment and tracrRNA), and/or the sequence encoding a Cas enzyme.

As used herein, the terms “under the control”, “under transcriptional control”, “operably positioned”, and “operably linked” mean that a promoter is in a correct functional location and or orientation in relation to a nucleic acid sequence, a DNA fragment, or a gene, to control transcriptional initiation and/or expression of that sequence, DNA fragment or gene.

The promoter may be constitutive, regulatable or inducible; cell type-specific, tissue-specific, or species-specific.

A constitutive promoter is an unregulated promoter that allows for continual transcription of the gene under the promoter's control. Many promoter/regulatory sequences useful for driving constitutive expression of a gene are available in the art and include, but are not limited to, for example, U6 (human U6 small nuclear promoter). H1 (human polymerase III RNA promoter), CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), and the like.

The present CRISPR component (e.g., a CRISPR guide sequence, a sequence encoding an sgRNA, a sequence encoding a chimeric RNA, a sequence encoding a crRNA, a sequence encoding a tracrRNA, a sequence encoding a Cas enzyme) may be under the control of an inducible promoter or a constitutive promoter.

The transcriptional activity of inducible promoters may be induced by chemical or physical factors. Chemically-regulated inducible promoters may include promoters whose transcriptional activity is regulated by the presence or absence of oxygen, a metabolite, alcohol, tetracycline, steroids, metal and other compounds. Physically-regulated inducible promoters, including promoters whose transcriptional activity is regulated by the presence or absence of heat, low or high temperatures, acid, base, or light. In one embodiment, the inducible promoter is pH-sensitive (pH inducible), lire inducer for the inducible promoter may be located in the biological tissue or environmental medium to which the composition is administered or targeted, or is to be administered or targeted. Examples of tissue specific or inducible promoter/regulatory sequences include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters can be found at http://www.invivogen.com/prom-a-list. In addition, promoters which can be induced in response to inducing agents such as metals, glucocorticoids, tetracycline, hormones, and the like, are also contemplated for use with the present systems and methods. The pH level of a particular biological tissue can affect the inducibility of the pH inducible promoter. See, for example, Boron, et al., Medical Physiology: A Cellular and Molecular Approach. Elsevier/Saunders. (2004), ISBN 1-4160-2328-3. Examples of inducers that can induce the activity of the inducible promoters also include, but are not limited to, doxycycline, radiation, temperature change, alcohol, antibiotic, steroid, metal, salicylic acid, ethylene, benzothiadiazole, or other compound. In an embodiment, the at least one inducer includes at least one of arabinose, lactose, maltose, sucrose, glucose, xylose, galactose, rhamnose, fructose, melibiose, starch, inunlin, lipopolysaccharide, arsenic, cadmium, chromium, temperature, light, antibiotic, oxygen level, xylan, nisin, L-arabinose, allolactose, D-glucose, D-xylose, D-galactose, ampicillin, tetracycline, penicillin, pristinamycin, retinoic acid, or interferon. Other examples of inducers include, but are not limited to, clathrate or caged compound, protocell, coacervate, microsphere, Janus particle, proteinoid, laminate, helical rod, liposome, macroscopic tube, niosome, sphingosome, vesicular tube, vesicle, unilamellar vesicle, multilamellar vesicle, multivesicular vesicle, lipid layer, lipid bilayer, micelle, organelle, nucleic acid, peptide, polypeptide, protein, glycopcptide, glycolipid, lipoprotein, lipopolysaccharide, sphingolipid, glycosphingolipid, glycoprotein, peptidoglycan, lipid, carbohydrate, metalloprotein, proteoglycan, chromosome, nucleus, acid, buffer, protic solvent, aprotic solvent, nitric oxide, vitamin, mineral, nitrous oxide, nitric oxide synthase, amino acid, micelle, polymer, copolymer, monomer, prepolymer, cell receptor, adhesion molecule, cytokine, chemokine, immunoglobulin, antibody, antigen, extracellular matrix, cell ligand, zwitterionic material, cationic material, oligonucleotide, nanotube, piloxymer, transfersome, gas, element, contaminant, radioactive panicle, radiation, hormone, virus, quantum dot, temperature change, thermal energy, or contrast agent. Theys, et al., Abstract, Curr. Gene Ther. vol. 3, no. 3 pp. 207-221 (2003).

### Arrangement of Elements

Each of the present constructs (or vectors) may contain at least two CRISPR guide sequences. The two or more CRISPR guide sequences may comprise two or more copies of a CRISPR guide sequence, two or more different CRISPR guide sequences, or combinations thereof.

The two or more CRISPR guide sequences may be operably linked to the same promoter or linked to different promoters. For example, the two or more CRISPR guide sequences may be operably linked to two or more promoters. In one embodiment, two CRISPR guide sequences are operably linked to two promoters; thus two transcripts (each comprising one gRNA) would be transcribed. In another embodiment, two CRISPR guide sequences are operably linked to a promoter; thus one transcript (comprising dual gRNAs) would be transcribed.

The two or more promoters of the present system may take suitable position and/or orientation. For example, the two or more promoters may be unidirectional or bidirectional.

The present system (e.g., the present library, constructs, vectors, etc.) driving expression of one or more elements of a CRISPR system may be introduced into a population of cells to target one or more target sites.

For example, a sequence encoding a Cas enzyme and a sequence encoding an sgRNA (or a sequence (or two sequences) encoding crRNA and tracrRNA which are expressed as two separate transcripts) are operably linked to separate promoters on separate vectors. Alternatively, a sequence encoding a Cas enzyme and a sequence encoding an sgRNA (or a sequence (or two sequences) encoding crRNA and tracrRNA which are expressed as two separate transcripts) are operably linked to separate promoters in a single vector.

The CRISPR system elements that are combined in a single vector may be arranged in any suitable orientation, such as one element located 5′ with respect to (upstream of) or 3′ with respect to (downstream of) a second element. The coding sequence of one element may be located on the same or opposite strand of the coding sequence of a second element, and oriented in the same or opposite direction.

In some embodiments, the present system (e.g., a vector, a construct, etc.) comprises: (a) at least one first promoter operably linked to one or more CRISPR guide sequences; (b) at least one second promoter operably linked to one or more CRISPR guide sequences; and (c) at least one third promoter operably linked to a sequence encoding a Cas enzyme.

In some embodiments, a single promoter drives expression of a transcript encoding a Cas enzyme and one or more of the CRISPR guide sequence, tracrRNA-binding segment (optionally operably linked to the CRISPR guide sequence), and a tracrRNA sequence. In some embodiments, a sequence encoding a Cas enzyme, one or more CRISPR guide sequences, a sequence encoding the tracrRNA-binding segment, and a sequence encoding the tracrRNA sequence are operably linked to and expressed from two or more promoters.

In some embodiments, a vector comprises one or more insertion sites, such as a restriction recognition site (also referred to as a restriction site, or a cloning site). One or more insertion sites (e.g. about or more than about 1, 2, 3, 4, 5, 6, 7, 8, 9, 10, or more insertion sites) may be located upstream and/or downstream of one or more sequences encoding one or more CRISPR components.

As used herein, “CRISPR components” refers to any of, gRNA, crRNA, a tracrRNA-binding segment, tracrRNA, sgRNA, chimeric RNA, and a Cas enzyme.

In some embodiments, a vector comprises one or more insertion sites upstream of a sequence encoding a tracrRNA-binding segment and/or a sequence encoding a tracrRNA, and/or a sequence encoding a chimeric RNA or an sgRNA. In some embodiments, a vector comprises one or more insertion sites downstream of a sequence encoding a tracrRNA-binding segment, and/or a sequence encoding a tracrRNA, and/or a sequence encoding a chimeric RNA or an sgRNA. In some embodiments, a vector comprises two or more insertion sites, each insertion site being located between two tracrRNA-binding segments so as to allow insertion of a CRISPR guide sequence at each site.

In some embodiments, a vector comprises an insertion site downstream of a promoter. In some embodiments, a vector comprises one or more insertion sites (e.g. about or more than about 1, 2, 3, 4, 5, 6, 7, 8, 9, 10, or more insertion sites) downstream of a promoter.

The CRISPR guide sequences and the sequence encoding a Cas enzyme may be located on the same or different vectors.

In some embodiments, sequences encoding one or more of the present CRISPR components are part of a vector system transiently transfected into the host cell. Alternatively or additionally, sequences encoding one or more of the present CRISPR components are stably integrated into a genome of a host cell.

When two or more different CRISPR guide sequences are used, a single expression construct may be used to target CRISPR activity to two or more different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a population of cells. U.S. Patent Publication No. 20150133315.

Alternatively, when two or more different CRISPR guide sequences are used, two or more vectors may be used to target CRISPR activity to two or more different, corresponding target sequences within a cell. For example, a single vector, or multiple vectors, may comprise about or more than about 1, 2, 3, 4, 5, 6, 7, 8, 9, 10, 15, 20, or more CRISPR guide sequences. In some embodiments, about or more than about 1, 2, 3, 4, 5, 6, 7, 8, 9, 10, or more such guide-sequence-containing vectors may be provided, and optionally delivered to a population of cells.

### Vectors

As used herein, a “vector” may be any of a number of nucleic acids into which a desired sequence or sequences may be inserted for transport between different genetic environments or for expression in a host cell. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art.

Vectors include, but are not limited to, viral vectors, plasmids, cosmids, fosmids, phages, phage lambda, phagemids, and artificial chromosomes.

Viral vectors may be derived from DNA viruses or RNA viruses, which have either episomal or integrated genomes after delivery to the cell. Anderson, Science 256:808-813 (1992); Nabel & Feigner, TIBTECH 11:211-217 (1993); Mitani & Caskey. TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

Viral vectors may be derived from retroviruses (including lentiviruses), replication defective retroviruses (including replication defective lentiviruses), adenoviruses, replication defective adenoviruses, adeno-associated viruses (AAV), herpes simplex viruses, and poxviruses. In some embodiments, the vector is a lentiviral vector. Options for gene delivery of viral constructs are known (see, e.g., Ausubel et al., Current Protocols in Molecular Biology, John Wiley & Sons, New York, 1989; Kay, M. A., et al., 2001 Nat. Medic. 7(1):33-40; and Walther W. and Stein U., 2000 Drugs, 60(2): 249-71).

Any subtype, serotype and pseudotype of lentiviruses, and both naturally occurring and recombinant forms, may be used as a vector for the present systems and methods. Lentiviral vectors may include, without limitation, primate lentiviruses, goat lentiviruses, sheep lentiviruses, horse lentiviruses, cat lentiviruses, and cattle lentiviruses.

The term AAV covers all subtypes, serotypes and pseudotypes, and both naturally occurring and recombinant forms. AAV viral vectors may be selected from among any AAV serotype, including, without limitation, AAV1, AAV2, AAV3, AAV4, AAV5, AAV6, AAV7, AAV8, AAV9, AAV10 or other known and unknown AAV serotypes. Pseudotyped AAV refers to an AAV that contains capsid proteins from one serotype and a viral genome of a second serotype.

A variety of vectors may be used to deliver CRISPR components to the targeted cells and/or a subject. In some embodiments, one or more of the present CRISPR components are part of the same vector, or two or more vectors.

The constructs encoding the present CRISPR components can be delivered to the subject using one or more vectors (e.g., 1, 2, 3, 4, 5, 6, 7, 8, 9, or more vectors). One or more CRISPR guide sequences can be packaged into a vector. A Cas enzyme can be packaged into the same, or alternatively separate, vectors.

Vectors may further contain one or more marker sequences suitable for use in the identification of cells which have or have not been infected, transformed, transduced or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein, red fluorescent protein). Molecular Cloning: A Laboratory Manual, Fourth Edition, Cold Spring Harbor Laboratory Press, 2012. Current Protocols in Molecular Biology, John Wiley & Sons, Inc.

Vectors can be designed for expression of CRISPR components in prokaryotic or eukaryotic cells. For example, CRISPR transcripts can be expressed in bacterial cells such as Escherichia coli, insect cells (using baculovirus expression vectors), yeast cells, or mammalian cells. Alternatively, one or mote of the CRISPR components can be transcribed and translated in vitro.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system).

In some embodiments, a prokaryote is used to amplify copies of a vector and express one or more nucleic acids, such as to provide a source of one or more proteins for delivery to a host cell or host organism. In some embodiments, a vector is a yeast expression vector. In some embodiments, a vector drives protein expression in insect cells using baculovirus expression vectors.

In some embodiments, a vector is capable of driving expression of one or more sequences in mammalian cells using a mammalian expression vector. Non-limiting examples of promoters include those derived from polyoma, adenovirus 2, cytomegalovirus, simian virus 40, and others disclosed herein and known in the art. Sambrook et al., Molecular Cloning: A Laboratory Manual, 4th ed., Cold Spring Harbor Laboratory, Cold Spring Harbor Laboratory Press, Cold Spring Harbor, N.Y., 2012.

Reporter genes that may be used with the present systems and methods include, but are not limited to, sequences encoding glutathione-S-transferase (GST), horseradish peroxidase (HRP), chloramphenicol acetyltransferase (CAT) beta-galactosidase, luciferase, green fluorescent protein (GFP), cyan fluorescent protein (CFP), yellow fluorescent protein (YFP), and auto fluorescent proteins including blue fluorescent protein (BFP), may be introduced into a cell to encode a gene product which serves as a marker.

In some embodiments, sequences encoding one or more of the present CRISPR components may contain modifications including, but not limited to, a 5′ cap (e.g., a 7-methylguanylate cap (m7G)); a 3′ polyadenylated tail (i.e., a 3′ poly(A) tail); a riboswitch sequence (e.g., to allow for regulated stability and or regulated accessibility by proteins and or protein complexes); a stability control sequence; a sequence that forms a dsRNA duplex (e.g., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, historic deacetylases, and the like); and combinations thereof.

### Libraries

The present disclosure also provides for libraries comprising two or more of the present constructs (e.g., vectors), or two or more of gRNAs. A library of constructs (e.g., vectors) refers to a collection of two or more constructs (e.g., vectors).

The present disclosure provides a library of gRNAs. The present disclosure provides a library of nucleic acids (e.g., constructs, vectors, etc.) encoding gRNAs. For example, the present library may be a library of CRISPR guide sequences. The present library may be a vector library encoding gRNAs.

In one embodiment, every pair of the genes of interest is knocked-out in parallel. In some embodiments, the present library is a dual-gRNA (pairwise) library. The library contains a plurality of vectors, each vector having two CRISPR guide sequences. The pairwise library can be used to generate libraries of multiple combination orders (e.g., tri-wise, quad-wise or more than quad-wise or n-wise combination). For example, an insert library can be generated, for example, by conducting PCR on the pairwise vector library. In a first combination event, all of the vectors can be paired with all of the inserts, generating a full combinatorial set of tri-wise or quad-wise combinations. This process may be iterative to generate libraries of higher combination orders.

The present system may be a genome wide library. The library may target a subset of the genome of an organism, or a set of genes relating to a particular pathway or phenotype. The set of genes targeted by the present system may be the entire genome of an organism, or may be a subset of the genome of an organism. The set of genes may relate to a particular pathway (for example, an enzymatic pathway, an immune pathway or a cell division pathway) or a particular disease or group of diseases or disorders (e.g., cancer) may be selected.

The present library may target about 100 or more sequences, about 1000 or more sequences or about 20,000 or more sequences, or the entire genome of an organism. The target sequences may be different loci within the same gene(s). The target sequences may be different genes. The present library may target 2 to 60 different loci within the same gene target or across multiple gene targets. For example, the present library may target 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 different DNA sequences. In some embodiments, the present library may target more than 60 different loci within the same gene target or across multiple gene targets, such as 65, 70, 75, 80, 85, 90, 95, 100 or more different DNA sequences.

The library may alter (decrease or increase) the expression level or the function of at least one gene, e.g., all genes of the set of genes. The library may result in a knockout of at least one gene, e.g., all genes of the set of genes.

The present system (e.g., libraries, constructs, vectors) and method may reduce (or increase) the expression level of at least one gene by at least 10%, 15%, 20%, 25%, 30%, 35%, 40%, 45%, 50%, 55%, 60%, or at least 65% as compared to expression level of the gene in the absence of the present system. The present system (e.g., the present library) may reduce activity of at feast one protein encoded by a gene by at least 10%, 15%, 20%, 25%, 30%, 35%, 40%, 45%, 50%, 55%, 60%, or at least 65% as compared to activity of the protein encoded by the gene in the absence of the present system.

The present disclosure also provides a cell library.

In one embodiment, for a set of genes, each cell may have a pair of genes knocked out, and the entire library of cells may have all of the genes knocked out in a pairwise fashion. In another embodiment, for a set of genes, each cell may have a group of three genes knocked out, and the entire library of cells may have all of the genes knocked out in a tri-wise fashion. In yet another embodiment, for a set of genes, each cell may have a group of four genes knocked out, and the entire library of cells may have all of the genes knocked out in a quad-wise fashion. In still another embodiment, for a set of genes, each cell may have a group of n genes knocked out, and the entire library of cells may have all of the genes knocked out in a n-wise fashion.

In one embodiment, for a set of genes, each cell may have the expression levels of a pair of genes altered (decreased or increased), and the entire library of cells may have the expression levels of all of the genes altered (decreased or increased) in a pairwise fashion. In another embodiment, for a set of genes, each cell may have the expression levels of a group of three genes altered (decreased or increased), and the entire library of cells may have the expression levels of all of the genes altered (decreased or increased) in a tri-wise fashion. In yet another embodiment, for a set of genes, each cell may have the expression levels of a group of four genes altered (decreased or increased), and the entire library of cells may have the expression levels of all of the genes altered (decreased or increased) in a quad-wise fashion. In still another embodiment, for a set of genes, each cell may have the expression levels of a group of n genes altered (decreased or increased), and the entire library of cells may have the expression levels of all of the genes altered (decreased or increased) in a n-wise fashion.

DNA may be isolated from cells by any method well known in the art. For example, DNA extraction may include two or more of the following steps: cell lysis, addition of a detergent or surfactant, addition of protease, addition of RNase, alcohol precipitation (e.g., ethanol precipitation, or isopropanol precipitation), salt precipitation, organic extraction (e.g., phenol-chloroform extraction), solid phase extraction, silica gel membrane extraction, CsC1 gradient purification. Various commercial kits (e.g., kits of Qiagen, Valencia, Calif.) can be used to extract DNA.

The DNA fragments may or may not be separated by gel electrophoresis prior to insertion into vectors.

DNA fragments may be inserted into vectors using, e.g., DNA ligase. Each vector may contain a different insert of DNA. In some embodiments, fragmented DNA is end-repaired before being ligated to a vector. Fragmented DNAs may be ligated to adapters before being inserted into vectors.

This present system (libraries, constructs, vectors, etc.) may be used for screening genetic interactions, gene functions, etc. in cellular processes as well as diseases.

A library may be introduced into a population of cells in vitro or in vivo to screen for beneficial mutations (or combinations of mutations) in a set of genes, and a desired phenotype identified. The set of genes may be the entire genome of an organism, a subset of the genome of an organism, or genes involved in target pathways (e g., a metabolic pathway, a signaling pathway, etc.).

### Introduction of Nucleic Acids Into Cells

The present disclosure also provides for a method of mapping genetic interactions by delivering the present system (e.g., the present libraries, constructs, vectors) into a population of cells. The present disclosure also provides for methods of delivering one or more nucleic acids (e.g., the present library, constructs, vectors), one or more transcripts thereof, and/or one or proteins transcribed therefrom, to a population of cells.

The present system (e.g., the present library) may also encode a Cas enzyme, such as a Cas9. The present method may also include delivering DNA or mRNA encoding a Cas enzyme to the cells. Alternatively or additionally, the cells may express a Cas enzyme (e.g., Cas9 expressing cells). For example, the cells may be stably transfected with DNA encoding Cas9. The cells may have DNA encoding Cas9 stably integrated. Expressing the nucleic acid molecule may also be accomplished by integrating the nucleic acid molecule into the genome. U.S. Patent Publication No. 20160186213.

In some embodiments, a Cas enzyme in combination with (and optionally complexed with) a gRNA or a CRISPR guide sequence is delivered to a cell.

The Cas enzyme (e.g., Cas9) may be driven by an inducible promoter (e.g. doxycycline inducible promoter) or a constitutive promoter.

Nucleic acids can be delivered as part of a larger construct, such as a plasmid or viral vector, or directly.

Nucleic acids can be introduced into a population of cells using methods and techniques that are standard in the art, such as infection, transformation, transfection, transduction etc. Non-limiting examples of methods to introduce nucleic acids into cells include lipofectamine transfection, calcium phosphate co-precipitation, electroporation, DEAE-dextran treatment, microinjection, viral infection, chemical transformation, electroporation, lipid vesicles, viral transporters, ballistic transformation, pressure induced transformation, viral transduction, particle bombardment, and other methods known in the art.

The nucleic acids may be delivered to cultured cells in vitro. Alternatively, the nucleic acids may be delivered to the cells in a subject. Cells may be isolated from a subject and modified using the present system and method in vitro.

The present disclosure further provides cells produced by the methods described herein, and organisms (such as animals, plants, or fungi) comprising or produced from such cells.

In some embodiments, a population of cells are transiently or non-transiently (e.g., stably) transfected or infected with one or more vectors described herein. In some embodiments, a population of cells are infected or transfected as it naturally occurs in a subject. In some embodiments, a population of cells that are infected or transfected are taken from a subject. In some embodiments, the cells are derived from cells taken from a subject, such as a cell line. Cell lines are available from a variety of sources known to those with skill in the art (see, e.g., the American Type Culture Collection (ATCC)). In some embodiments, a cell infected or transfected with one or more vectors described herein is used to establish a cell line comprising one or more sequences encoding one or more of the present CRISPR components.

Suitable cells include, but are not limited to, mammalian cells (e.g., human cells, mouse cells, rat cells, etc.), primary cells, stem cells, avian cells, plant cells, insect cells, bacterial cells, fungal cells (e.g., yeast cells), and any other type of cells known to those skilled in the art.

### Screening

The present disclosure encompasses assaying or screening cells expressing the present system (e.g., libraries, constructs, vectors).

The ability of a guide sequence to direct sequence-specific binding of a CRISPR complex to a target sequence may be assessed by any suitable assay, such as by Surveyor assay.

Surveyor assay detects mutations and polymorphisms in a DNA mixture. Surveyor Nuclease can be a member of the CEL family of mismatch-specific nucleases derived from celery. Surveyor Nuclease recognizes and cleaves mismatches due to the presence of single nucleotide polymorphisms (SNPs) or small insertions or deletions. Surveyor nuclease cleaves with high specificity at the 3′ side of any mismatch site in both DNA strands, including all base substitutions and insertion/deletions up to at least 12 nucleotides. Surveyor nuclease technology involves four steps: (i) PCR to amplify target DNA from the cell or tissue samples underwent Cas9 nuclease-mediated cleavage (here we expect to see an nonhomogeneous or mosaic pattern of nuclease treatment on cells, some cells got cuts, some cells don't); (ii) hybridization to form heteroduplexes between affected and unaffected DNA (Because the affected DNA sequence will be different from the affected, a bulge structure resulted from the mismatch can form after denature and renature); (iii) treatment of annealed DNA with Surveyor nuclease to cleave heteroduplexes (cut the bulges), and (iv) analysis of digested DNA products using the detection/separation platform of choice, for instance, agarose gel electrophoresis. The Cas9 nuclease-mediated cleavage efficacy can be estimated by the ratio of Surveyor nuclease-digested over undigested DNA. Surveyor mutation assay kits are commercially available from Integrated DNA Technologies (IDT), Coraville, Iowa.

Similarly, cleavage of a target sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other suitable assays are also possible.

To determine the function of the genes modulated by the present CRISPR-Cas system, cells contacted with the present system are compared to control cells, e.g., without the CRISPR-Cas system or with a non-specific CRISPR-Cas system, to examine the extent of modification (e.g., inhibition or activation) of gene activity, and/or change (e.g., increase or decrease) in gene expression level. Control samples may be assigned a relative gene expression value of 100%. The present CRISPR-Cas system may decrease or increase gene activity and/or gene expression level by about or at least about 80%, 50%, 25%, 10%, 5%, 2-fold, 5-fold, 10-fold, 20-fold, at least about 1.2 fold, at least about 1.4 fold, at least about 1.5 fold, at least about 1.8 fold, at least about 2 fold, at least about 3 fold, at least about 4 fold, at least about 5 fold, at least about 6 fold, at least about 7 fold, at least about 8 fold, at least about 10 fold, at least about 15 fold, at least about 20 fold, at least about 25 fold, at least about 30 fold, at least about 35 fold, at least about 40 fold, at least about 50 fold, at least about 60 fold, at least about 70 fold, at least about 80 fold, at least about 90 fold, at least about 100 fold, at least about 200 fold, at least about 250 fold, at least about 300 fold, at least about 400 fold, or at least about 500 fold, compared to the gene expression level and/or gene activity in the control.

The expression level of the modified gene may be at least about 1.2 fold, at least about 1.4 fold, at least about 1.5 fold, at least about 1.8 fold, at least about 2 fold, at least about 3 fold, at least about 4 fold, at least about 5 fold, at least about 6 fold, at least about 7 fold, at least about 8 fold, at least about 10 fold, at least about 15 fold, at least about 20 fold, at least about 25 fold, at least about 30 fold, at least about 35 fold, at least about 40 fold, at least about 50 fold, at least about 60 fold, at least about 70 fold, at least about 80 fold, at least about 90 fold, at least about 100 fold, at least about 200 fold, of the expression level of the gene in its natural form (e.g., in control cells).

For example, an assay is used to determine whether or not the gene targeting is associated with a selected phenotype. It can be determined whether two or more genes are associated with the same phenotype. The present system (e.g., libraries, constructs, vectors) can also be used to determine whether a gene participates with other genes in a particular phenotype.

A phenotype refers to any phenotype, e.g., any observable characteristic or functional effect that can be measured in an assay such as changes in cell growth, proliferation, morphology, enzyme function, signal transduction, expression patterns, downstream expression patterns, reporter gene activation, hormone release, growth factor release, neurotransmitter release, ligand binding, apoptosis, and product formation. A candidate gene is “associated with” a selected phenotype if modulation of gene expression of the candidate gene causes a change in the selected phenotype.

In certain embodiments, gene expression and/or modification can be assayed by determining any parameter that is indirectly or directly affected by the expression of the target genes. Such parameters include, e.g., changes in RNA or protein levels, changes in RNA stability, changes in protein activity, changes in product levels, changes in downstream gene expression, changes in reporter gene transcription or expression (e.g., via chemiluminescence, fluorescence, calorimetric reactions, antibody binding, inducible markers, ligand binding assays, such as assaying luciferase, CAT, beta-galactosidase, beta-glucuronidase, GFP (see, e.g., Mistili & Spector, Nature Biotechnology 15:961-964 (1997)); changes in signal transduction, changes in phosphorylation and/or dephosphorylation, changes in receptor-ligand interactions, changes in second messenger (such as cGMP and inositol triphosphate (IP3)) concentrations, changes in cell growth, changes in intracellular calcium levels; changes in cytokine release, and changes in neovascularization, etc., as described herein. These assays can be in vitro, in vivo, and ex vivo.

Such assays include, e.g., transformation assays, e.g., changes in proliferation, anchorage dependence, growth factor dependence, foci formation, growth in soft agar, tumor proliferation in nude mice, and tumor vascularization in nude mice; apoptosis assays, e.g., DNA laddering and cell death, expression of genes involved in apoptosis; signal transduction assays, e.g., changes in intracellular calcium, cAMP, cGMP, IP3, changes in hormone and neurotransmitter release; receptor assays, e.g., estrogen receptor and cell growth; growth factor assays, e.g., EPO, hypoxia and erythrocyte colony forming units assays; enzyme product assays, e.g., FAD-2 induced oil desaturation; transcription assays, e.g., reporter gene assays; and protein production assays, e.g., VEGF ELISAs.

The present functional screens allow for discovery of novel human and mammalian therapeutic applications, including the discovery of novel drugs, for, e.g., treatment of genetic diseases, cancer, fungal, protozoal, bacterial, and viral infection, ischemia, vascular disease, arthritis, immunological disorders, etc.

In some embodiments, cells transiently or non-transiently transfected or infected with one or more vectors described herein, or cell lines derived from such cells are used in assessing one or more test compounds.

The present methods and systems can be used for mapping genetic interactions, large-scale phenotyping, gene-to-function mapping, meta-genomic analysis, drug screening, disease diagnosis, prognosis, etc. WO2015071474.

The present methods and systems may be used to select cells with the modified genes (e.g., knockouts) that survive under a selective pressure. The method may contain the following steps: (a) contacting a population of cells with the present system (e.g., the present library, constructs, vectors, etc.); (b) optionally selecting for successfully infected or transfected cells; (c) applying the selective pressure; and (d) selecting the cells that survive under the selective pressure.

The selective pressure may be an application of a drug, FACS sorting of cell markers or aging.

The present methods and systems may be used to identify the genetic basis of one or more medical symptoms exhibited by a subject, lire method may contain the following steps: (a) obtaining a biological sample from the subject and isolating a population of cells having a first phenotype from the biological sample; (b) contacting the cells having the first phenotype with the present system (e.g., the present library); (c) optionally selecting for successfully infected or transfected cells; (d) applying the selective pressure; (e) selecting the cells that survive under the selective pressure; (f) determining the genes that interact with the first phenotype and identifying the genetic basis of the one or more medical symptoms exhibited by the subject.

The present methods and systems may be used in functional genomic screens for, e.g., assaying whether knockout of each pair of genes confers a survival advantage under the selective pressure of a screen, and/or predicting whether a drug (e.g., a chemotherapeutic agent) is effective.

The present methods and vectors can be used to identify two or more inhibitors targeting two or more genes. The inhibitors can be used to treat disorders or diseases.

For example, the inhibitors identified by the present method may be used to reduce or inhibit cell proliferation. The cell may be a cancer cell. The inhibitors may be used to treat cancer. In some embodiments, the inhibitors are a CRISPR guide sequence (or a gRNA); an antisense RNA, an siRNA or shRNA; and a small molecule.

The present application provides methods for treating a disorder (e.g., cancer, or other disorders described herein) in a subject comprising administering to the subject a combination of two or more inhibitors targeting two or more genes. The inhibitors are administered in a therapeutically effective amount.

In some embodiments, the effective amount of each of the two or more inhibitors administered in the combination is less than the effective amount of the inhibitor when not administered in the combination.

The present methods and systems may be used for CRISPR display which is a targeted localization method that uses Sp. Cas9 to deploy large RNA cargos to DNA loci. For example, one or more RNA domains may be inserted into one or more gRNAs. In some embodiments, the vector encodes a gRNA fused to one or more RNA domain. In some embodiments, the RNA is a non-coding RNA or fragment thereof. In such embodiments, the RNA domain may be targeted to a DNA loci. Shechner et al., CRISPR Display: a modular method for locus-specific targeting of long noncoding RNAs and synthetic RNA devices in vivo, Nature Methods, 2015, 12(7):664-670.

### Kits

The present disclosure also encompasses kits containing the present systems (e.g., the present library, constructs, vectors).

In some embodiments, the kit comprises a vector system and instructions for using the kit. Elements may be provided individually or in combinations, and may be provided in any suitable container, such as a vial, a bottle, or a tube.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form).

### DNA Sequencing

The present systems may be analyzed by sequencing or by microarray analysis. It should be appreciated that any means of determining DNA sequence is compatible with identifying one or more DNA elements.

The DNA may be extracted and sequenced to identify CRISPR guide sequences and/or genetic modifications.

DNA may be amplified via polymerase chain reaction (PCR) before being sequenced.

The DNA may be sequenced using vector-based primers; or a specific gene is sought by using specific primers. PCR and sequencing techniques are well known in the art; reagents and equipment are readily available commercially.

Non-limiting examples of sequencing methods include Sanger sequencing or chain termination sequencing, Maxam-Gilbert sequencing, capillary array DNA sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nat. Biotechnol., 16:381-384 (1998)), and sequencing by hybridization (Chee et al., Science, 274:610-614 (1996); Drmanac et al., Science, 260:1649-1652 (1993); Drmanac et al., Nat. Biotechnol., 16:54-58 (1998)), NGS (next-generation sequencing) (Chen et al., Genome Res. 18:1143-1149 (2008): Srivatsan et al. PloS Genet 4:e1000139 (2008)), Polony sequencing (Porreca et al., Curr. Protoc. Mol. Biol. Chp. 7; 7.8 (2006), ion semiconductor sequencing (Elliott et al., J. Biomol Tech. 1:24-30 (2010), DNA nanoball sequencing (Kaji et al., Chem Soc Rev 39:948-56 (2010), single-molecule real-time sequencing (Flusberg et al., Nat. Methods 6:461-5 (2010), sequencing by synthesis (e.g., Illumina/Solexa sequencing), sequencing by ligation, sequencing by hybridization, nanopore DNA sequencing (Wanunu, Phys Life Rev 9:125-58 (2012), massively Parallel Signature Sequencing (MPSS); pyro sequencing. SOLiD sequencing (McKeman et al. 2009 Genome Res 19:1527-1541; Shearer et al. 2010 Proc Natl Acad Sci USA 107:21104-21109); shortgun sequencing; Heliscope single molecule sequencing; single molecule real time (SMRT) sequencing. U.S. Patent Publication No. 20140329705.

High-throughput sequencing, next-generation sequencing (NGS), and/or deep-sequencing technologies include, but are not limited to, Illumina/Solex sequencing technology (Bentley et al. 2008 Nature 456:53-59), Roche/454 (Margulies et al. 2005 Nature 437:376-380). Pacbio (Flusberg et al. 2010 Nature methods 7:461-465; Korlach et al. 2010 Methods in enzymology 472:431-455; Schadt et al. 2010 Nature reviews. Genetics 11:647-657; Schadt et al. 2010 Human molecular genetics 19:R227-240; Eid et al. 2009 Science 323:133-138; Imelfort and Edwards, 2009 Briefings in bioinformatics 10:609-618), Ion Torrent (Rothberg et al. 2011 Nature 475:348-352)) and more. For example, Polony technology utilizes a single step to generate billions of “distinct clones” for sequencing. As another example, ion-sensitive field-effect transistor (JSFET) sequencing technology provides a non-optically based sequencing technique U.S. Patent Publication No. 20140329712.

Several methods of DNA extraction and analysis are encompassed in the present disclosure. As used herein “deep sequencing” indicates that the depth of the process is many times larger than the length of the sequence under study. Deep sequencing is encompassed in next generation sequencing methods which include but are not limited to single molecule realtime sequencing (Pacific Bio), Ion semiconductor (Ion torrent sequencing). Pyrosequencmg (454), Sequencing by synthesis (lilumina), Sequencing by ligations (SOLID sequencing) and Chain termination (Sanger sequencing).

Sequencing of the DNA after introduction of the present system (e.g., the present library) into cells can identify the specific genes (e.g., the specific pair(s) of genes) affected by the CRISPR guide sequences corresponding to a selected phenotype.

Sequencing reads may be first subjected to quality control to identify overrepresented sequences and low-quality ends. The start and/or end of a read may or may not be trimmed. Sequences mapping to the genome may be removed and excluded from further analysis. As used herein, the term “read” refers to the sequence of a DNA fragment obtained after sequencing. In certain embodiments, the reads are paired-end reads, where the DNA fragment is sequenced from both ends of the molecule.

### Organisms

The organism may be a eukaryotic organism, including human and non-human eukaryotic organisms. The organism may be a multicellular eukaryotic organism. The organism may be an animal, for example a mammal such as a mouse, rat, or rabbit. Also, the organism may be an arthropod such as an insect. The organism also may be a plant or a fungus. The organism may be prokaryotic.

In one embodiment, the cell is a mammalian cell, such as a human cell. Human cells may include human embryonic kidney cells (e.g., HEK293T cells), human dermal fibroblasts, human cancer cells, etc. The organism may be a mammal, such as humans, dogs and cats, farm animals such as cows, pigs, sheep, horses, goats and the like, and laboratory animals (e.g., rats, mice, guinea pigs, and the like). The present system may be delivered by plasmids or delivered by viruses such as lentiviruses, adenoviruses or AAVs.

In another embodiment, the cell is a yeast cell. The organism may be a yeast. The present system may be delivered by plasmids or shuttle vectors. In yet another embodiment, the cell is a bacterial cell. The organism may be bacteria. The present system may be delivered by plasmids or phages.

The following are examples of the present invention and are not to be construed as limiting.

### EXAMPLES

### Example 1

The CRISPR (Clustered Regularly interspaced Short Palindromic Repeats) system is a sequence-specific nuclease system (Wiedenheft, B. et al. Nature 482, 331-338 (2012); Jinek, M. et al. Science 337, 816-821 (2012); Mali, P. et al. Science 339, 823-826 (2013); Cong, L. et al. Science 339, 819-823 (2013)). The CRISPR system exploits RNA-guided DNA-binding and sequence-specific cleavage of target DNA. The guide RNA/Cas combination confers site specificity to the nuclease. A crRNA contains nucleotides that are complementary to a target DNA sequence which may be upstream of a genomic PAM (protospacer adjacent motifs) site and a constant RNA scaffold region. The Cas (CRISPR-associated) protein binds to the crRNA/tracrRNA and the target DNA to which the crRNA/tracrRNA binds and introduces a double-strand break in a defined location upstream of the PAM site. Cas9 harbors two independent nuclease domains homologous to HNH and RuvC endonucleases, and by mutating either of the two domains, the Cas9 protein can be converted to a nickase that introduces single-strand breaks (Cong. L. et al. Science 339, 819-823 (2013)).

To extend CRISPR-Cas9 approaches for high-throughput combinatorial studies of genetic interactions, a general strategy is needed to interrogate pairs of chromosomal loci in a streamlined systematic and facile manner. Here, we describe the development of a multiplex strategy for assessing genetic interactions using CRISPR-Cas9 (MoSAIC).

### MATERIALS AND METHODS

### Cell Culture

HEK 293T cells were obtained from the American Tissue Collection Center (ATCC) and grown at 37° C., 5% CO₂ in high-Glucose Dulbecco's modified Eagle's medium (DMEM) containing 10% fetal bovine serum and 1% Penicillin/Streptomycin (Life Technologies). 293FT cells were obtained from Life Technologies and were maintained in the same medium formulation and supplemented with 0.1 mM nonessential amino acids, 2 mM L-glutamine and 500 ug/ml Geneticin.

Lentivirus production and transduction.

Lentivirus was produced in 293FT cells and stable Cas9-eGFP cells were transduced as previously described (Broad Institute RNAi Consortium; http://www broadinstitute.org/rnai/public/resources/protocols).

Generation of Inducible eGFP-Cas9 Cell Line

Briefly, doxycycline hyclate (Sigma) inducible Cas9 cells were generated as follows. 293T cells were infected with lentiviral particles carrying the pCW-Cas9 construct (Addgene 50661) at MOI of 0.3 followed by clonal selection. We selected a clone with highest differential Cas9 expression following 48-hour induction using immunostaining of FLAG-tagged Cas9, followed by flow cytometry.

The eGFP-Cas9 clone was infected with lentivirus containing gRNA constructs targeting eGFP and STAT1 or eGFP-only. Twenty-four hours post-infection, the media was changed and supplemented with 10 ug/ml blasticidin (Life Technologies) and cells were selected for three days, prior to doxycycline induction of Cas9. Cells were harvested on days 14, 21, and 28 post-induction. Gene knockout efficiencies were measured by either flow cytometry or SURVEYOR assay. Flow cytometry was performed using a LSRII or LSR Fortessa to quantify fraction of eGFP positive cells.

### MoSAIC Vector Construction

MV1, MV.3, MV.5, MV.6 and MV.7 originated from lentivector v_w0, originally called plxsgRNA (Addgene 50662). A point mutation was made in the PGK promoter to eliminate the BsmB1 restriction site for all down-stream cloning (v_w0). MV.2 originated from pLenticrispr (49535). Vs.d1 was amplified with primers containing eGFP gRNA 1/STAT1 gRNA2 and cloned into the pLenticrispr vector to generate an all-in-one vector containing two gRNAs. To clone MV.1 backbone, pLenticrispr was used as a template with vs_p39(f) and vs_40(r) to amplify an insert containing the reverse direction chimeric RNA, filler region with BsmB1 restriction sites and a forward direction chimeric RNA sequence. The chimeric-filler-chimeric was cloned into v_w0. To clone in gRNAs, vs.d5 (dsDNA) containing reverse direction H1 promoter. LoxP site and forward direction U6 promoter, was amplified with primers containing eGFP gRNA 1 and STAT1 gRNA 2 as well as BsmB1 restriction sites. The PCR product containing both gRNAs and both promoters was cloned into the MV.1 backbone to generate MV.1.1 and MV.1.2.

To clone MV.3 backbone, H1 promoter expressing short tracrRNA was cloned into v_w0 from px261 (Addgene 42337). To clone in gRNAs, vs.d11 (containing U6 promoter) was amplified with primers vs_p79 and vs_p80/vs_p81/vs_p82 and PCR products were cloned into MV.3 backbone.

To clone MV.5 backbone, the U6 promoter, filler region and chimeric RNA was cloned into v_w0 from lenticrispr_v1 (Addgene #49535) using vs_p59/vs_p40 primers. To clone in gRNAs, v_w2 containing chimeric RNA-LoxP-site and H1 promoter were amplified with primers containing eGFP gRNA 1 and STAT1 gRNA 2 as well as BsmB1 restriction sites.

To clone MV.6 backbone, U6 promoter, the filler region with BsmB1 restriction sites and the chimeric RNA v2, was cloned into v_w0 using vs_d10. To clone in gRNAs, v_w2 containing chimeric RNA-loxp-site and H1 promoter were amplified with primers containing eGFP gRNA 1 and STAT1 gRNA 2 as well as BsmB1 restriction sites.

In MV.7, used the backbone established in MV.6. To clone in gRNAs, oligo pairs (vs_p75/vs_p76 and vs_p77/vs_p78) containing-BsmB1 Overhang-gRNA1-chimeric RNA-gRNA 2 were synthesized, annealed and ligated into backbone.

### DNA Constructs Used

Unless noted, all DNA constructs and primers were obtained from IDT (Geneblocks) and used for PCR and assembly steps as described above.

vs_d1: chimeric RNA-LoxP site-U6 promoter (SEQ ID NO: 1):
GTTTTAGAGCTAGAAATAGCAAGTTAAAATAAGGCTAGTCCGTTATCAACTTGAAAAAGTGGCACCGAGTCGGTGC
TTTTTTATAACTTCGTATAGCATACATTATACGAAGTTATGAGGGCCTATTTCCCATGATTCCTTCATATTTGCAT
ATACGATACAAGGCTGTTAGAGAGATAATTAGAATTAATTTGACTGTAAACACAAAGATATTAGTACAAAATACGT
GACGTAGAAAGTAATAATTTCTTGGGTAGTTTGCAGTTTTAAAATTATGTTTTAAAATGGACTATCATATGCTTAC
CGTAACTTGAAAGTATTTCGATTTCTTGGCTTTATATATCTTGTGGAAAGGACGAAACACC
vs_d5: reverse H1 promoter-LoxP site-U6 promoter forward (SEQ ID NO: 2):
TAGATCTGTGGTCTCATACAGAACTTATAAGATTCCCAAATCCAAAGACATTTCACGTTTATGGTGATTTCCCAGA
ACACATAGCGACATGCAAATATTGCAGGGCGCCACTCCCCTGTCCCTCACAGCCATCTTCCTGCCAGGGCGCACGC
GCGCTGGGTGTTCCCGCCTAGTGACACTGGGCCCGCGATTCCTTGGAGCGGGTTGATGACGTCAGCGTTCGAATTA
TAACTTCGTATAGCATACATTATACGAAGTTATGAGGGCCTATTTCCCATGATTCCTTCATATTTGCATATACGAT
ACAAGGCTGTTAGAGAGATAATTAGAATTAATTTGACTGTAAACACAAAGATATTAGTACAAAATACGTGACGTAG
AAAGTAATAATTTCTTGGGTAGTTTGCAGTTTTAAAATTATGTTTTAAAATGGACTATCATATGCTTACCGTAACT
TGAAAGTATTTCGATTTCTTGGCTTTATATATCTTGTGGAAAGGACGAAACACC
v_w2: chimeric RNA-LoxP site-forward H1 promoter (SEQ ID NO: 3):
GTTTTAGAGCTAGAAATAGCAAGTTAAAATAAGGCTAGTCCGTTATCAACTTGAAAAAGTGGCACCGAGTCGGTGC
TTTTTTATAACTTCGTATAGCATACATTATACGAAGTTATAATTCGAACGCTGACGTCATCAACCCGCTCCAAGGA
ATCGCGGGCCCAGTGTCACTAGGCGGGAACACCCAGCGCGCGTGCGCCCTGGCAGGAAGATGGCTGTGAGGGACAG
GGGAGTGGCGCCCTGCAATATTTGCATGTCGCTATGTGTTCTGGGAAATCACCATAAACGTGAAATGTCTTTGGAT
TTGGGAATCTTATAAGTTCTGTATGAGACCACAGATCTA
vs_dI0: Xhol site-forward U6 promoter-BsmB1 filler region (36nt)-Chimeric
RNA version 2-Nhel (SEQ ID NO: 4):
ATTCGAACTCGAGGAGGGCCTATTTCCCATGATTCCTTCATATTTGCATATACGATACAAGGCTGTTAGAGAGATA
ATTAGAATTAATTTGACTGTAAACACAAAGATATTAGTACAAAATACGTGACGTAGAAAGTAATAATTTCTTGGGT
AGTTTGCAGTTTTAAAATTATGTTTTAAAATGGACTATCATATGCTTACCGTAACTTGAAAGTATTTCGATTTCTT
GGCTTTATATATCTTGTGGAAAGGACGAAACACCGGAGACGGTTTTCTTGCTCTTTTTTGTACGTCTCTGTTTTAG
AGCCGGAAACGGCAAGTTAAAATAAGGCTAGTCCGTTATCAACTTGAAAAAGTGGCACCGAGTCGGTGCTTTTTTG
CTAGCGCTAAC
vs_d11: forward U6 promoter (SEQ ID NO: 5):
GAGGGCCTATTTCCCATGATTCCTTCATATTTGCATATACGATACAAGGCTGTTAGAGAGATAATTAGAATTAATT
TGACTGTAAACACAAAGATATTAGTACAAAATACGTGACGTAGAAAGTAATAATTTCTTGGGTAGTTTGCAGTTTT
AAAATTATGTTTTAAAATGGACTATCATATGCTTACCGTAACTTGAAAGTATTTCGATTTCTTGGCTTTATATATC
TTGTGGAAAGGACGAAACACC
Primers Used
vs_p26 (forward sequencing primer, all) (SEQ ID NO: 6):
AATGGACTATCATATGCTTACCGTAACTTGAAAGTATTTCG
vs_p64 (reverse sequencing primer, MV.6/MV.7) (SEQ ID NO: 7):
TATTTTAACTTGCCGTTTCCGGC
vs_p59 (forward) (SEQ ID NO: 8):
ACGGACTCGAGGAGGGCCTATTTCCCATGATTC
vs_p40 (reverse) (SEQ ID NO: 9):
GATCACGGAGCTAGCCTGCCATTTGTCTCAAGATCTAGAATTC
vs_p75 (SEQ ID NO: 10):
CACCGGAGCTGGACGGCGACGTAAAGTTTTAGAGCTAGAAATAGCAAGTTAAAATAAGGCTAGTCCGTTATCAACT
TGAAAAAGTGGCACCGAGTCGGTGCTTGAAGTTCGAGGGCGACACCC
vs_p76 (SEQ ID NO: 11):
AAACGGGTGTCGCCCTCGAACTTCAAGCACCGACTCGGTGCCACTTTTTCAAGTTGATAACGGACTAGCCTTATTT
TAACTTGCTATTTCTAGCTCTAAAACTTTACGTCGCCGTCCAGCTCC
vs_p77 (SEQ ID NO: 12):
CACCGGAGCTGGACGGCGACGTAAAGTTTTAGAGCTAGAAATAGCAAGTTAAAATAAGGCTAGTCCGTTATCAACT
TGAAAAAGTGGCACCGAGTCGGTGCTTTCCCCGGGGAAGTTCGAGGGCGACACCC
vs_p78 (SEQ ID NO: 13):
AAACGGGTGTCGCCCTCGAACTTCCCCGGGGAAAGCACCGACTCGGTGCCACTTTTTCAAGTTGATAACGGACTAG
CCTTATTTTAACTTGCTATTTCTAGCTCTAAAACTTTACGTCGCCGTCCAGCTCC
vs_p79 (forward) (SEQ ID NO: 14):
AGGGATCCTGAGGGCCTATTTCCCATGA
vs_p80 (reverse) (SEQ ID NO: 15):
GCGCTAGCTAAAAACAGCATAGCTCTAAAACGGGTGTCGCCCTCGAACTTCACAGCATAGCTCTAAAACTTTACGT
CGCCGTCCAGCTCGGTGTTTCGTCCTTTCCACAA
vs_p81 (reverse) (SEQ ID NO: 16):
TTAGCGCTAGCTAAAAGTTTTGGGACCATTCAAAACAGCATAGCTCTAAAACGGGTGTCGCCCTCGAACTTCGTTT
TGGGACCATTCAAAACAGCATAGCTCTAAAACTTTACGTCGCCGTCCAGCTCGGTGTTTCGTCCTTTCCACAAG
vs_p82 (reverse) (SEQ ID NO: 17):
AGCGCTAGCTAAAAGTTTTGGGACCATTCAAAACAGCATAGCTCTAAAACGGGTGTCGCCCTCGAACTTCGTTTTG
GGACCATTCAAAACAGCATAGCTCTAAAACTTTACGTCGCCGTCCAGCTCGTTTTGGGACCATTCAAAACAGCATA
GCTCTAAAACGGTGTTTCGTCCTTTCCACA
gRNA Sequence Used
eGFP gRNA 1 (SEQ ID NO: 18):
GAGCTGGACGGCGACGTAAA
eGFP gRNA 2 (SEQ ID NO: 19):
GAAGTTCGAGGGCGACACCC
STAT1 gRNA 1 (SEQ ID NO: 20):
GATCATCCAGCTGTGACAGG
STAT1 gRNA 2 (SEQ ID NO: 21):
CCTGTCACAGCTGGATGATC
eGFP sequence (SEQ ID NO: 22):
ATGGTGAGCAAGGGCGAGGAGCTGTTCACCGGGGTGGTGCCCATCCTGGTCGAGCTGGACGGCGACGTAAACGGCC
ACAAGTTCAGCGTGTCCGGCGAGGGCGAGGGCGATGCCACCTACGGCAAGCTGACCCTGAAGTTCATCTGCACCAC
CGGCAAGCTGCCCGTGCCCTGGCCCACCCTCGTGACCACCCTGACCTACGGCGTGCAGTGCTTCAGCCGCTACCCC
GACCACATGAAGCAGCACGACTTCTTCAAGTCCGCCATGCCCGAAGGCTACGTCCAGGAGCGCACCATCTTCTTCA
AGGACGACGGCAACTACAAGACCCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAACTACAACAGCCACAACGTCTAT
ATCATGGCCGACAAGCAGAAGAACGGCATCAAGGTGAACTTCAAGATCCGCCACAACATCGAGGACGGCAGCGTGC
AGCTCGCCGACCACTACCAGCAGAACACCCCCATCGGCGACGGCCCCGTGCTGCTGCCCGACAACCACTACCTGAG
CACCCAGTCCGCCCTGAGCAAAGACCCCAACGAGAAGCGCGATCACATGGTCCTGCTGGAGTTCGTGACCGCCGCC
GGGATCACTCTCGGCATGGACGAGCTGTACAAGTAA

### Modeling of Cas9-gRNA-Chimeric-RNAv2 Complex

We used the crystal structure of the sgRNA-targetDNA-cas9 complex to model the UA31CG-AU32GC-sgRNA [17], The corresponding base pairs (U31-A38 and A32-U37) were mutated with 3DNA keeping the sugar-phosphate backbone conformation and the base reference frame as in the crystal structure [18]. The mutated RNA structure was then locally minimized with NAMD through the autoIMD plugin [19]. Atoms from mutated nucleotides were free to move while atoms within 8 Å of any mutated nucleotide atom were fixed and the remaining atoms were excluded during minimization. Conjugate gradient minimization was carried out using the CHARMM27 force-field during 15,000 steps (time-step 2 fs).

### RESULTS

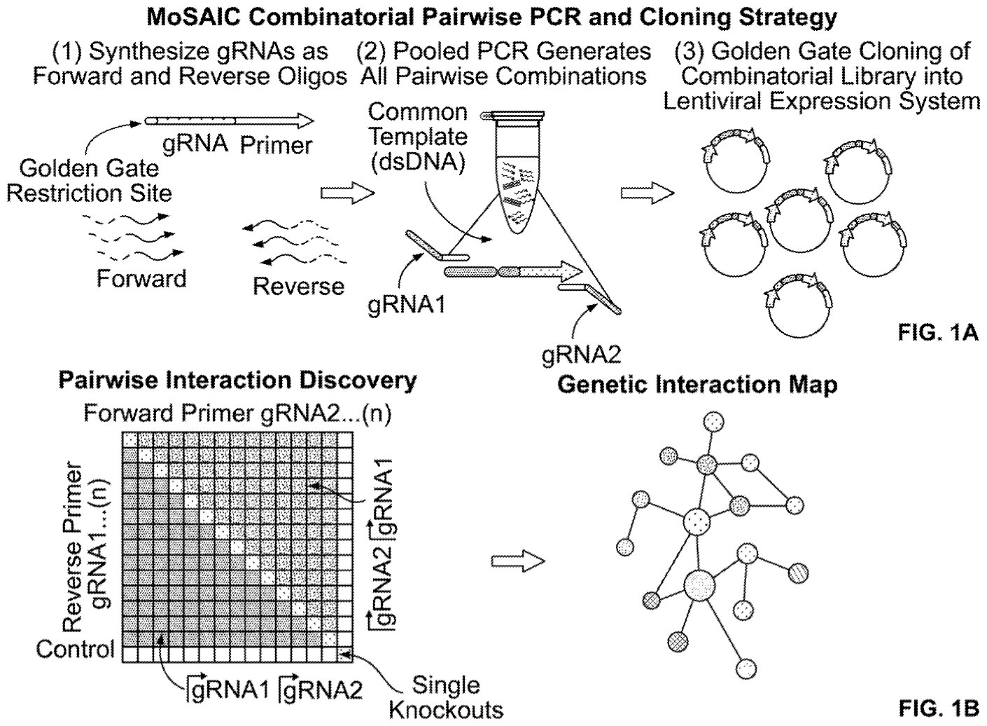

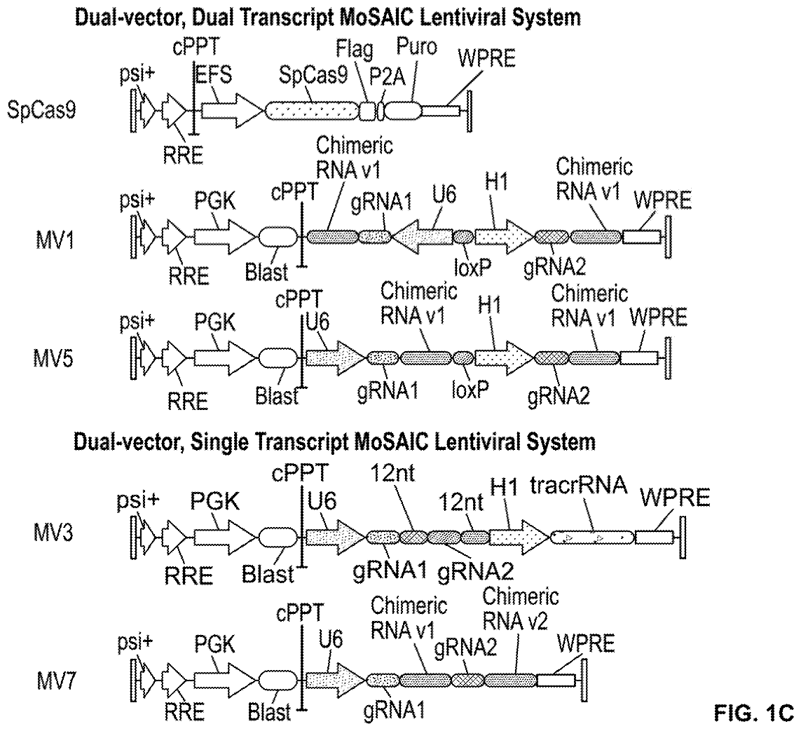

In order to comprehensively map genetic interactions in a gene network, all possible single and double knockouts (KO) need to be simultaneously interrogated. MoSAIC achieves this in a single step through PCR of a common DMA template with gRNA primer pools (FIG. 1A). The first position gRNAs act as the forward primers while the second position gRNAs act as the reverse primers The pooled PCR product is then cloned into a lentiviral expression vector resulting in an exhaustive combinatorial dual-gRNA library. In addition to directing genome editing to the desired targets, lentiviral integration of each gRNA pair in the library serves as a unique molecular barcode of each mutant for subsequent multiplex interrogation of the cell population (FIG. 1B).

To optimize the system for simultaneous targeting of Cas9 to multiple loci, we designed and tested two MoSAIC-compatible strategies: 1) dual promoter, dual gRNA transcripts, and 2) single promoter, single RNA transcript (dual gRNA fusion). We explored several designs that use RNA Pol III promoters U6 and H1 in different positions and orientations (FIG. 1C), having eliminated designs where the common templates contain sequences that result in DNA hairpins; for example, inward facing promoters would necessitate a common template containing two complimentary chimeric RNAs. After lentiviral transduction of MoSAIC designs into HEK293T test inducible Cas9 cells containing an integrated eGFP, we monitored Cas9-mediated eGFP KO by flow-cytometry 14 and 21 days post Cas9 induction (see Materials and Methods).

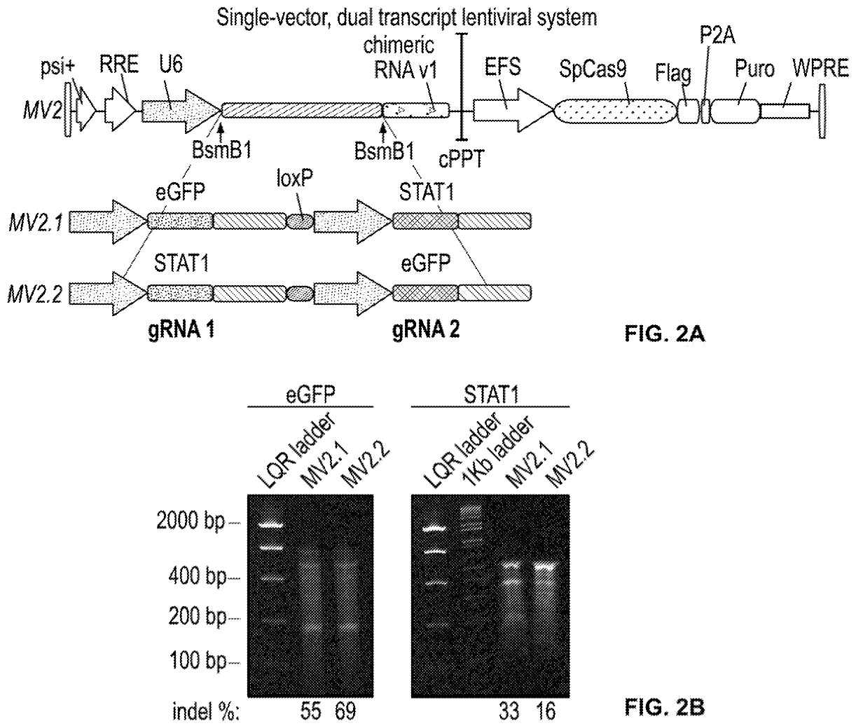

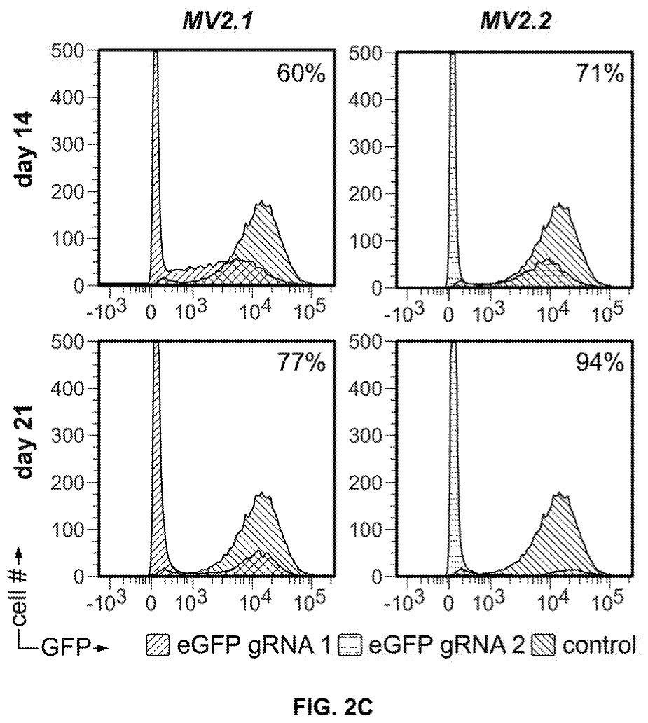

We began by benchmarking a previously described approach utilizing two unidirectional U6 promoters to express dual gRNAs [13] (designs MV2; FIG. 2A). We found that gRNAs expressed from the first U6 position resulted in lower efficiency than those expressed from the second position, irrespective of the target gene. Targeted KO efficiency for eGFP was determined by SURVEYOR assay to be 55% and 69% for the first and second gRNA positions respectively after 14 days (FIG. 2B). Similarly, KO efficiency for STAT1 was 16% and 33% for the first and second gRNA positions. Flow cytometry measurements of eGFP KO at day 14 show consistent trends of higher second gRNA position KO efficiency. The positional KO efficiency bias persists even as the overall KO efficiency improves for both positions beyond day 21 (FIG. 2C).

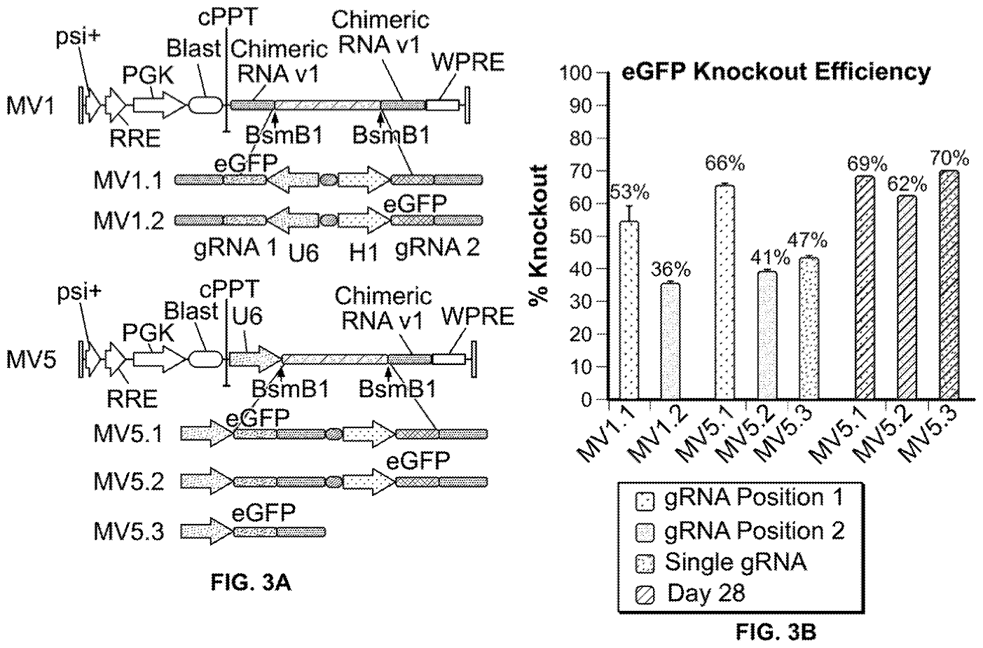

We then explored whether the promoter choice and orientation impacted KO efficiency in a position-dependent fashion (designs MV1 and MV5; FIG. 3A). For the bidirectional U6-H1 design (MV1), the first gRNA position driven by the U6 promoter showed higher efficiency compared to the second gRNA position driven by the H1 promoter (53% vs. 36%; FIG. 3B). For the unidirectional U6-H1 design (MV5), we observed KO efficiencies of 66% and 41% at the first gRNA position (U6 promoter) and second position (H1 promoter) respectively (FIG. 3B) at day 14. These results suggest that H1 promoter may be a weaker promoter (in general or transiently at day 14) and in the second gRNA position in contrast to the dual U6 promoter findings (FIG. 2). However, the KO efficiencies for MV5 designs in gRNA position 1 and 2 eventually converge to 69% and 62% respectively by day 28 (FIG. 3B), showing that the KO efficiency from the H1 promoter eventually reach that of the U6 promoter. The single gRNA control using the U6 promoter also reaches similar KO efficiency (70%) after 28 days. Together, these data highlight the significant impact of promoter position and orientation on KO efficiency for dual gRNA expression and suggest that the unidirectional U6-H1 design (MV5) is the most optimal implementation for targeting Cas9 to multiple loci.

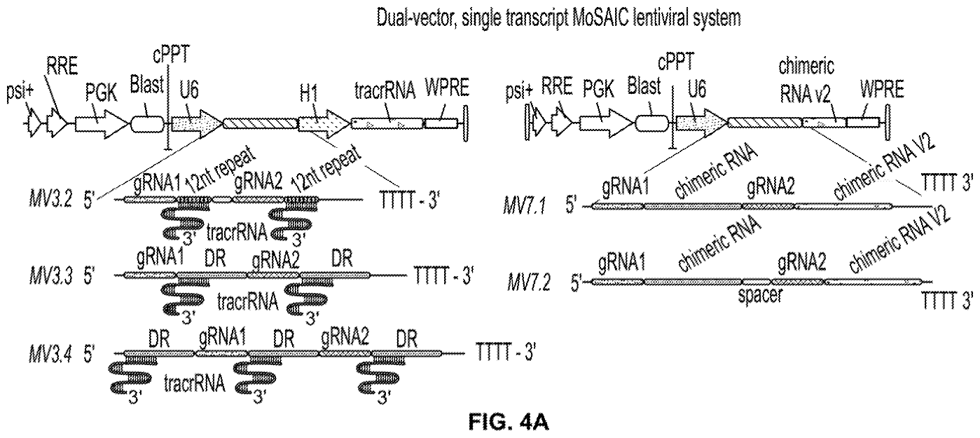

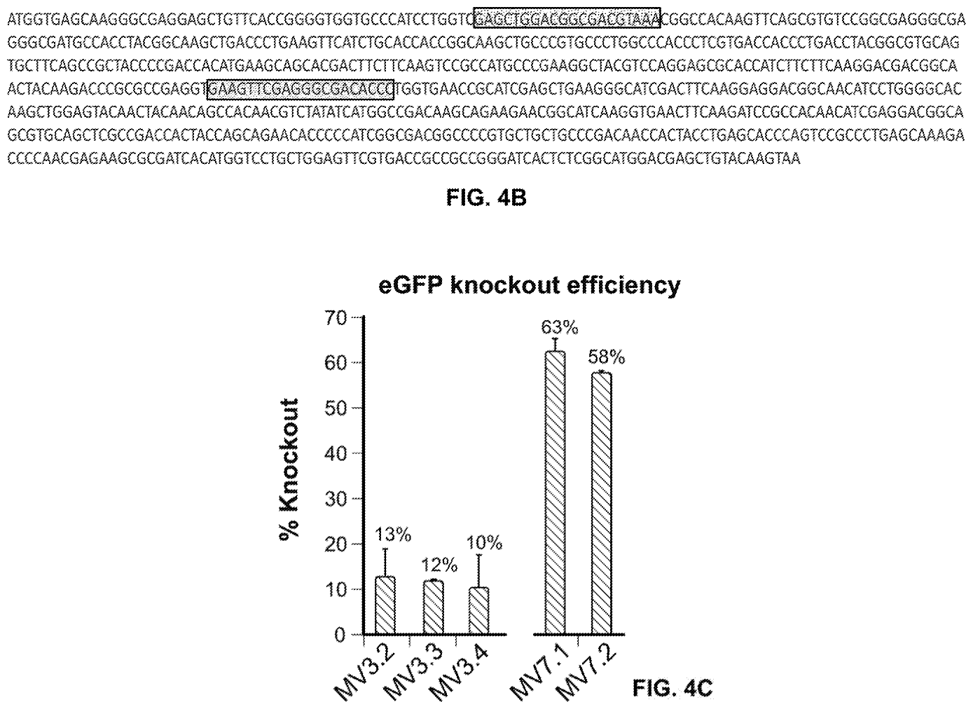

Previously, studies demonstrated that targeting Cas9 to multiple loci could be achieved by co-expressing the RNA cleavage enzyme Csy4 along with multiplexed gRNA expression from single RNA transcripts containing RNA cleavage sites [20]. Another study observed that flanking each gRNA with S. pyogenes direct repeat (DR) sequences is sufficient for multiplexed Cas9-mediated KO in the absence of the SpRNase III RNA cleavage enzyme [15]. In order to increase the multiplexing potential of MoSAIC, we explored whether single RNA transcripts encoding multiple gRNAs can lead to efficient Cas9 targeting and gene KO. Four RNA transcript designs (each MoSAIC compatible for pairwise combinatorial library assembly) driven by a single U6 promoter and targeting two positions of an integrated eGFP gene were tested (MV3, MV7; FIG. 4). In the MV3 designs, a tracrRNA was expressed separately from an H1 promoter in place of the chimeric RNA. Repeat regions between two gRNAs were altered to contain either a 12 bp sequence complementary to the tracrRNA (MV3.2) or the DR sequence (MV3.3 and MV3.4) previously described [15] (FIG. 4A). We observed that designs using DR sequences (MV3.3 and MV3.4) led to a limited KO efficiency (12% and 10% respectively), in accordance with previous findings. The reduced DR sequence consisting of only the 12 bp repeat region (MV3.2) lead to KO efficiency on par with constructs MV3.3 and MV3.4 containing the full DR sequences (FIG. 4C). While it remains to be elucidated, these results suggest that RNA cleavage of multi-gRNA transcripts may not be necessary for Cas9 mediated gene editing.

We further explored MV7 designs that incorporated transcripts containing two tandem gRNA-chimeric RNA sequences and thus did not require a tracrRNA (FIG. 4A). This design lead to a KO efficiency (63%) that was as good as, if not better than, the dual promoter designs (FIG. 4C vs. FIG. 3B). When combined with dual U6-H strategies, MV7 designs may provide opportunities to increase editing efficiency (by encoding multiple gRNAs on a single transcript), as well as to reduce off-target editing (if used with nickase-Cas9) [21].

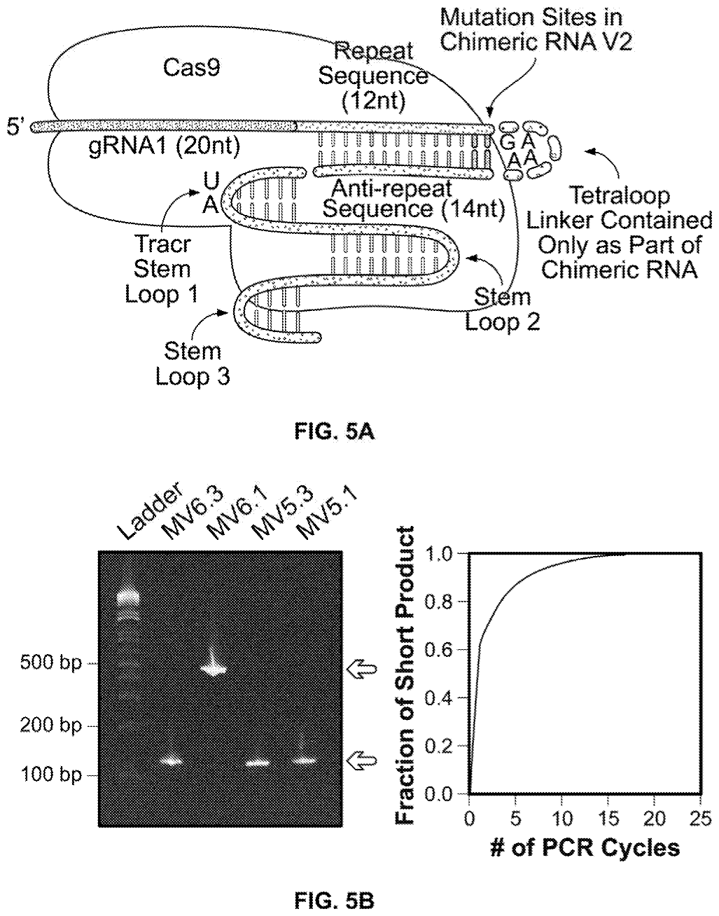

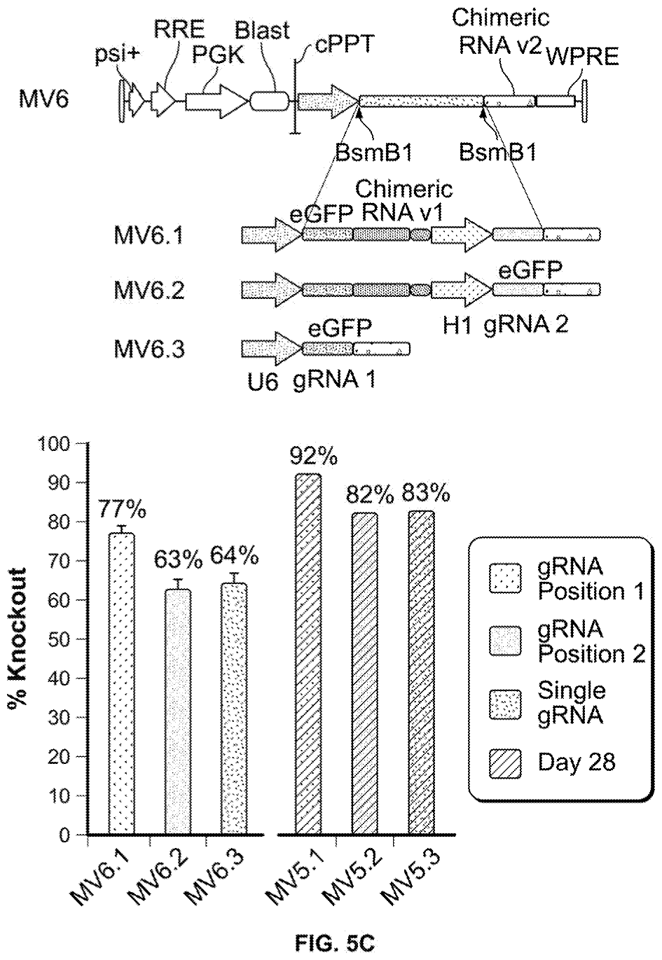

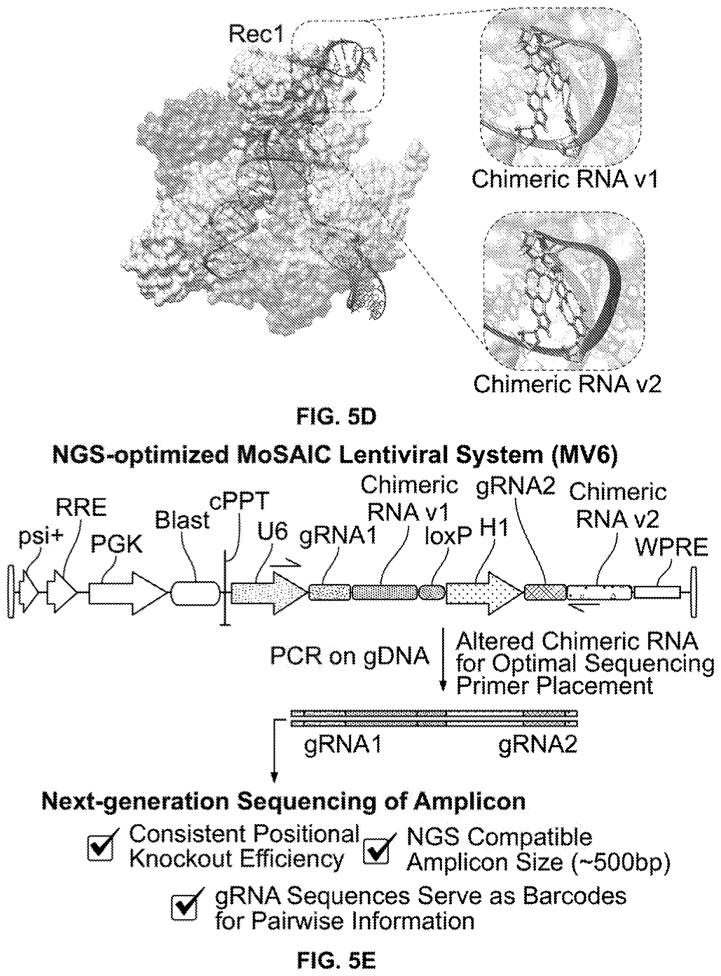

MoSAIC is designed such that gRNA pairs serve as barcodes that can then be PCR amplified and identified using next-generation sequencing. We achieved this by altering the second chimeric RNA sequence such that placement of a reverse sequencing primer results in PCR amplification of both gRNAs with an amplicon size that is NGS compatible (FIG. 5A). Primer placement at repeat regions, such as two U6 promoters or two identical chimeric RNAs leads to two potential PCR products, a long and a short, and favors the short product, which contains only one gRNA sequence (FIG. 5B). We utilized the S. pyogenes CRISPR-Cas9 crystal structure [17] to predict mutations in the chimeric RNA sequence that would not interfere with Cas9 function and allow for optimal primer placement (see Methods). Nucleotide positions 11-12 and 17-18 (corresponding to the repeat-anti-repeat duplex flanking the tetraloop) of wildtype chimeric RNA were altered from TA-TA to CG-CG to generate an altered orthogonal chimeric RNA sequence (v2) that is compatible with PCR of gRNA barcodes.

Indeed, the altered chimeric RNA enables recovery of full-length dual-gRNA barcode amplicon from extracted genomic DNA (FIG. 5B). We then measured the efficiency of Cas9-mediated KO using the altered chimeric RNA designs (MV6.2/MV6.3, FIG. 5C) and found that there is a significantly higher KO efficiency than the original (MV5.2/MV5.3) chimeric RNA (FIG. 3B). This increased efficiency may be the result of tetranucleotide stabilization due to an increase in intra-strand Hydrogen-bonding, which further stabilizes Rec1-RNA interactions within Cas9 (FIG. 5D), and raises the possibility that additional chimeric RNA variants may exist that lead to more efficient Cas9 editing. Importantly, these alterations enable a dual gRNA vector that is compatible with high-throughput screening (FIG. 5E).

### DISCUSSION

MoSAIC overcomes several key technical hurdles associated with high throughput generation and measurement of dual loci perturbations in mammalian cells.

We find that gRNA pairs expressed from dual U6-H1 promoters lead to optimal Cas9-mediated genome editing, which can be combined with single transcript multiple gRNA designs (MV7) to increase editing efficiency. The development of chimeric RNA variants that are compatible with pooled barcode amplification enable multiplex assessment of cell populations using NGS. Furthermore, this strategy is compatible with the use of different Cas9 variants, including CRISPRi [22] and CRISPRa [23], enabling both loss and gain-of-function combinatorial screens. Additionally, to facilitate subsequent iterative introduction of gRNA constructs and enable higher-order combinatorial genetic perturbations, the integrated lentiviral vector design includes loxP “landing-pad” sequences. The MoSAIC system advances the therapeutic potential of combinatorial Cas9-mediated genome editing and represents an important step towards comprehensive delineation of genetic networks relevant to human disease as well as fundamental aspects of cellular life.

### REFERENCES

1. Boone C, Bussey H, Andrews B J. Exploring genetic interactions and networks with yeast. Nat Rev Genet. 2007; 8(6):437-49. doi: 10.1038/nrg2085. PubMed PMID. 17510664.
2. Tong A H, Lesage G, Bader G D, Ding H, Xu H, Xin X, et al. Global mapping of the yeast genetic interaction network. Science. 2004; 303(5659):808-13. doi: 10.1126/science. 1091317. PubMed PMID: 4764870.
3. Butland G, Babu M, Diaz-Mejia J J, Bohdana F, Phanse S, Gold B, et al. eSGA: E. coli synthetic genetic array analysis. Nat Methods. 2008; 5(9):789-95. doi:10.1038/nmeth. 1239. PubMed PMID: 18677321.
4. Bassik M C, Kampmann M, Lebbink R J, Wang S, Hein M Y, Poser I, et al. A systematic mammalian genetic interaction map reveals pathways underlying ricin susceptibility. Cell. 2013; 152(4):909-22. doi: 10.1016/j.cell.2013.01.030. PubMed PMID: 23394947; PubMed Central PMCID: PMC3652613.
5. Hsu P D, Lender E S, Zhang F. Development and applications of CRISPR-Cas9 for genome engineering. Cell 2014; 157(6):1262-78. doi: 10.1016/j.cell2014.05.010. PubMed PMID: 24906146; PubMed Central PMCID: PMC4343198.
6. Mali P, Esvelt K M, Church G M. Cas9 as a versatile tool for engineering biology. Nat Methods, 2013; 10(10):957-63. doi: 10.1038/nmeth.2649. PubMed PMID. 24076990; PubMed Central PMCID: PMC4051438.
7. Doudna J A, Charpentier E. Genome editing. The new frontier of genome engineering with CRISPR-Cas9. Science. 2014:346(6213): 1258096. doi: 10.1126/science.1258096. PubMed PMID: 25430774.
8. Sander J D, Joung J K. CRISPR-Cas systems for editing, regulating and targeting genomes. Nat Biotechnol. 2014; 32(4):347-55. doi: 10.1038/nbt.2842. PubMed PMID: 24584096; PubMed Central PMCID: PMC4022601.
9. Shalem O, Sanjana N E, Hartenian E, Shi X, Scott D A, Mikkelsen 446 TS, et al. Genome447 scale CRISPR-Cas9 knockout screening in human cells. Science. 2014; 343(6166):84-7. doi: 10.1126/science.1247005. PubMed PMID: 24336571; PubMed Central PMCID: PMC4089965.
10. Zhou Y, Zhu S, Cai C, Yuan P, Li C, Huang Y, et al. High-throughput screening of a CRISPR-Cas9 library for functional genomics in human cells Nature. 2014; 509(7501):487-91. doi: 10.1038/nature 13166. PubMed PMID: 24717434.
11. Koike-Yusa H, Li Y, Tan E P, Velasco-Herrera Mdel C, Yusa K. Genome-wide recessive genetic screening in mammalian cells with a lentiviral CRISPR-guide RNA library. Nat Biotechnol. 2014; 32(3):267-73. doi: 10.1038/nbt.2800. PubMed PMID: 24535568.
12. Wang T, Wei J J, Sabatini D M, Lander E. S. Genetic screens in human cells using the CRISPR-Cas9 system. Science. 2014; 343(6166):80-4. doi: 10.1126/science. 1246981. PubMed PMID: 24336569; PubMed Central PMCID: PMC3972032.
13. Sakuma T, Nishikawa A, Kume S. Chayama K, Yamamoto T. Multiplex genome engineering in human cells using all-in-one CRISPR/Cas9 vector system. Sci Rep. 2014; 4:5400. doi. 10.1038/srep05400. PubMed PMID: 24954249; PubMed Central PMCID: PMC4066266.
14. Kabadi A M, Ousterout D G, Hilton I B, Gersbach C A. Multiplex CRISPR/Cas9-based genome engineering from a single lentiviral vector. Nucleic Acids Res. 2014; 42(19):e147. doi: 10.1093/nar/gku749. PubMed PMID: 25122746; PubMed Central PMCID: PMC4231726.
15. Cong L. Ran FA , Cox D, Lin S, Barretto R, Habib N, et al. Multiplex genome engineering using CRISPR/Cas systems. Science 2013; 339(6121):819-23. doi: 10.1126/science.1231143. PubMed PMID. 23287718; PubMed Central PMCID: PMC3795411.
16. Wong et al. Multiplexed barcoded CRISPR-Cas9 screening enabled by CombiGEM. Proc Natl Acad Sci USA. 2016; 113(9) 2544-9. doi: 10.1073/pnas.117883113.
17. Nishimasu H, Ran F A, Hsu P D. Konermann S, Shehata S I , Dohmae N, et al. Crystal structure of Cas9 in complex with guide RNA and target DNA. Cell. 2014; 156(5):935-49. doi. 10.1016/j.cell.2014.02.001.
18. Lu X J, Olson W K. 3DNA: a software package for the analysis, rebuilding and visualization of three-dimensional nucleic acid structures. Nucleic Acids Res. 2003; 31(17):5108-21. PubMed PMID: 12930962; PubMed Central PMCID: PMC212791.
19. Phillips J C, Braun R, Wang W, Gumbart J, Tajkhorshid E, Villa E, et al. Scalable molecular dynamics with NAMD. J Comput Chem. 2005:26(16); 1781-802. doi:10.1002/jcc.20289. PubMed PMID: 16222654; PubMed Central PMCID: PMC2486339.
20. Nissim L, Perli S D, Fridkin A, Perez-Pinera P, Lu T K. Multiplexed and programmable regulation of gene networks with an integrated RNA and CRISPR/Cas toolkit in human cells. Mol Cell. 2014; 54(4):698-710. doi; 10.1016/j.molcel.2014.04.022. PubMed PMID: 24837679; PubMed Central PMCID: PMC4077618.
21. Ran F A, Hsu P D, Lin C Y, Gootenberg J S, Konermann S. Trevino A E, et al. Double nicking by RNA-guided CRISPR Cas9 for enhanced genome editing specificity. Cell. 2013; 154(6): 1380-9. doi: 10.1016-j.cell.2013.08.021. PubMed PMID. 23992846; PubMed Central PMCID: PMC3856256.
22. Qi L S, Larson M H, Gilbert L A, Doudna J A, Weissman J S, Arkin A P, et al. Repurposing CRISPR as an RNA-guided platform for sequence-specific control of gene expression. Cell, 2013; 152(5):1173-83. doi: 10.1016/j.cell.2013.02.022. PubMed PMID: 23452860; PubMed Central PMCID: PMC3664290.
23. Gilbert L A, Horlbeck M A, Adamson B, Villalta J E, Chen Y, 497 Whitehead E H, et al. Genome-Scale CRISPR-Mediated Control of Gene Repression and Activation. Cell. 2014; 159(3):647-61. doi: 10.1016/j.cell.2014.09.029. PubMed PMID: 25307932; PubMed Central PMCID: PMC4253859.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

What is claimed is:

1. A method of constructing a guide RNA (gRNA) library targeting a set of genes, the method comprising the steps of:
(a) Providing a plurality of forward primers and a plurality of reverse primers, each forward primer comprising at least one CRISPR guide sequence targeting at least one gene of the set of genes, each reverse primer comprising at least one CRISPR guide sequence targeting at least one gene of the set of genes, wherein the plurality of forward primers comprises CRISPR guide sequences targeting all genes of the set of genes, wherein the plurality of reverse primers comprises CRISPR guide sequences targeting all genes of the set of genes, and wherein the CRISPR glide sequence encodes a guide RNA (gRNA); and
(b) Conducting PCR reactions using the plurality of forward primers and the plurality of reverse primers.

2. The method of claim 1, targeting the set of genes in a pairwise fashion, wherein each forward primer comprises one CRISPR guide sequence targeting one gene of the set of genes, and wherein each reverse primer comprises one CRISPR guide sequence targeting one gene of the set of genes.

3. The method of claim 1, further comprising a step (c) cloning the PCR products into a plurality of vectors.

4. The method of claim 3, wherein the vectors are viral vectors.

5. The method of claim 4, wherein the viral vectors are lentiviral vectors.

6. The method of claim 3, wherein after step (c) each vector encodes a CRISPR RNA (crRNA) and a trans-activating crRNA (tracrRNA), and wherein the crRNA comprises the gRNA.

7. The method of claim 6, wherein the crRNA and the tracrRNA are expressed as separate transcripts

8. The method of claim 6, wherein the crRNA and the tracrRNA are expressed as a single-guide RNA (sgRNA).

9. The method of claim 1, wherein expression of the CRISPR guide sequences is under the control of U6 promoter, H1 promoter, T7 promoter, or a combination thereof.

10. The method of claim 3, wherein each vector further encodes a Cas enzyme.

11. The method of claim 10, wherein the Cas enzyme is Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, orthologs thereof, or modified versions thereof.

12. The method of claim 10, wherein the Cas enzyme is Cas9.

13. The method of claim 10, wherein the Cas enzyme is under the control of an inducible promoter.

14. The method of claim 10, wherein the Cas enzyme comprises one or more mutations.

15. The method of claim 1, wherein the gRNA library alters function of at least one gene of the set of genes.

16. The method of claim 1, wherein the gRNA library alters expression of at least one gene of the set of genes.

17. The method of claim 1, wherein the gRNA library decreases expression of at least one gene of the set of genes by CRISPR interference (CRISPRi).

18. The method of claim 1, wherein the gRNA library increases expression of at least one gene of the set of genes by CRISPR activation (CRISPRa).

19. The method of claim 1, wherein the gRNA library results in a knockout of at least one gene of the set of genes.

20. The method of claim 19, wherein the gRNA library results in a knockout of all genes of the set of genes in pairs.

21. The method of claim 1, wherein the set of genes comprises entire genome or a subset of the genome of an organism.

22. The method of claim 21, wherein the organism is a human.

23. The method of claim 3, wherein the vectors further comprise a selection marker anchor a reporter gene.

24. A method of mapping genetic interactions, the method comprising the step of delivering a gRNA library constructed by the method of claim 1 into a population of cells.

25. The method of claim 24, wherein the cells express a Cas enzyme.

26. The method of claim 24, further comprising delivering DNA or mRNA encoding a Cas enzyme to the cells.

27. The method of claims 25 or 26, wherein the Cas enzyme is Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, orthologs thereof, or modified versions thereof.

28. The method of claim 27, wherein the Cas enzyme is Cas9.

29. The method of claim 24, wherein the gRNA library alters function of at least one gene of the set of genes in the cells.

30. The method of claim 24, wherein the gRNA library alters expression of at least one gene of the set of genes in the cells.

31. The method of claim 24, wherein the gRNA library decreases expression of at least one gene of the set of genes in the cells by CRISPR interference (CRISPRi).

32. The method of claim 24, wherein the gRNA library increases expression of at least one gene of the set of genes in the cells by CRISPR activation (CRISPRa).

33. The method of claim 24, wherein the gRNA library results in a knockout of at least one gene of the set of genes in the cells.

34. The method of claim 33, wherein the gRNA library results in a knockout of all genes of the set of genes in pairs.

35. The method of claim 33, wherein the knockcut is confirmed by sequencing.

36. The method of claim 35, wherein the sequencing is next-generation sequencing (NGS).

37. A gRNA library constructed by the method of claim 1.

38. A population of eukaryotic cells comprising a gRNA library constructed by the method of claim 1.

39. A kit comprising a gRNA library constructed by the method of claim 1.

40. A gRNA library targeting entire genome or a subset of the genome of an organism in a pairwise fashion, the library comprising a plurality of vectors, wherein each vector comprises at least two CRISPR guide sequences that target at least two genes of the organism, and wherein the library targets in parallel every pair of genes of the entire genome or a subset of the genome of the organism.
